(19) United States
(12) Patent Application Publication
(10) Pub. No.: US 2003/0199945 A1
(43) Pub. Date: Oct. 23, 2003

(54) Device and method for treating disordered breathing

(76) Inventor: James Ciulla, Paris, IL (US)

Correspondence Address: E. VICTOR INDIANO, INDIANO, VAUGHAN & ROBERTS, P.A., SUITE 850, ONE NORTH PENNSYLVAINA STREET, INDIANAPOLIS, IN, 46204, US

(21) Appl. No.: 10/364,625

(22) Filed: Feb. 10, 2003

Related U.S. Application Data
(60) Provisional application No. 60/356,258, filed on Feb. 11, 2002.
(60) Provisional application No. 60/373,294, filed on Apr. 16, 2002.

(51) Int. Cl. A61N 1/18

(52) U.S. Cl. 607/48

(57) ABSTRACT

A device is disclosed for treating sleep and breathing disorders of a patient, along with the method of using the device. The device includes a processor for receiving sensor inputs, processing the received sensor inputs, and generating commands through output devices. A first sensor is positionable for receiving breathing sound information emitted from one of the mouth and nose of a patient. The second sensor is positionable on a patient for receiving breathing sounds information from a patient's chest cavity. A third sensor is positionable for receiving information relating to the amount of chest expansion of a patient. A first output device is provided that is capable of providing an auditory signal to a patient. A second output device is capable of providing an electrical signal to a muscle group of a patient that simulates a human touching event. The first, second and third sensors, and the first and second output devices are operatively coupled to the processor to permit the processor to receive information input from the sensors, process the input information to the detect the existence of a sleep-breathing disorder event, and to generate command to at least one of the first and second output devices. The command is capable of directing the at least one output device to provide a series of progressively intrusive stimuli designed to condition the patient to terminate the sleep breathing disorder event, and ultimately, return to a more normal sleep pattern.

### I. CLAIM OF PRIORITY

The instant Application claims priority to James M. Ciulla U.S. Provisional Patent Applications Nos. 60/356,258 filed Feb. 11, 2002 and 60/373,294 filed Apr. 16, 2002.

### II. TECHNICAL FIELD OF THE INVENTION

The present invention relates to medical devices, and more particularly to an improved method and device for detecting and treating and sleep disorders and most particularly to treating sleep disordered breathing type sleep disorders such as sleep apnea.

### III. BACKGROUND OF THE INVENTION

A. Overview of Sleep Disorders

A variety of various sleep disordered breathing type disorders exist, the most common of which are a group of disorders referred to “sleep apnea” type disorders. Sleep apnea is a disorder of breathing during sleep. Typically, sleep apnea is accompanied by loud snoring by the patient. For one suffering from sleep apnea, sleep usually consists of brief periods throughout the night during which “apnea” events occur, wherein breathing stops. The result of sleep apnea is that people with sleep apnea generally do not get enough oxygen during sleep.

There are two primary types of sleep apnea, including obstructive sleep apnea, and central sleep apnea. Obstructive sleep apnea is the most common type of sleep apnea and is due to an obstruction in the throat during sleep. Persons who are bed partners of the apnea-suffering person will often notice pauses in the apnea patient's breathing, that can last for somewhere between 10 to 60 seconds between loud snores. These obstructions in the throat that cause the apnea result in a narrowing of the upper airway. This narrowing can be the result of several factors including inherent physical factors, such as the morphology of a particular patient's throat and breathing structures, excess weight and alcohol consumption before sleep.

Obstructive sleep apnea syndrome is characterized by repetitive episodes of upper airway obstruction that occur during sleep. These episodes are usually associated with a reduction of blood oxygen saturation. In other words, the airway becomes obstructed at several possible sites, which results in the user becoming hypoxic. The upper airway can be obstructed by one or more of a variety of factors including excess tissue in the airway, large tonsils and/or a large tongue, and usually includes the airway muscles relaxing and collapsing while asleep.

Another site of obstruction can be the nasal passages. Sometimes, the structure of the jaw and the airway can be a factor in causing sleep apnea.

Central sleep apnea is characterized by a cessation of breath due to a lack of effort in breathing during sleep. It is believed that the cessation is caused by a delay in the signal from the patient's brain to those motor functions that control breath. Central sleep apnea is not as common as obstructive sleep apnea, and is often more difficult to diagnose. Typically, central sleep apnea results from type of neuromuscular problems, but other sources can be the cause.

The symptoms of sleep apnea will vary from patient to patient. Sleep apnea patients often exhibit sleepiness during the day. At night, a sleep apnea patient will stop breathing frequently, which is an event about which the sleep apnea patient is usually unaware.

Unfortunately, the effects of sleep apnea are not confined to those described above. Some manifestations of obstructive sleep apnea including loud snoring that may upset the patients bed partner, along with morning headaches. Other manifestations include such things as high blood pressure, a dry mouth, easy weight gain, depression, difficulty concentrating, excess perspiration during sleep, heart burn, reduced libido, insomnia, frequent trips to the bathroom during the night, restless sleep and rapid weight gain. In summary, the affects of sleep apnea go far beyond the annoying snoring that serves to keep many a bed partner awake at night.

Even a cursory review of the medical literature reveals that all forms of Sleep Disordered Breathing have very significant and serious consequences on long and short-term morbidities and mortality. Obstructive Sleep Apnea Hypopnea Syndromes (OSAHS), Upper Airway Resistance Syndrome (UARS), Cheyne Stokes Respiration, Hypoventilation Syndromes (Pickwickian and Neuromuscular caused forms), Central Apnea and Mixed Syndromes, including COPD-OSA overlap syndrome, are increasingly being implicated in causing or aggravating multiple chronic degenerative processes.

Cardiovascular disease (including hypertension, Coronary Artery disease, Left Ventricular Hypertrophy, Congestive Heart Failure, arrhythmias, and strokes), CNS degeneration with memory loss, depression and personality changes, endocrine malfunctions (such as diabetes, increasing obesity, testosterone, growth hormone and possibly thyroid deficiency), morning headaches, Excessive Daytime Sleepiness and increased rates of serious accidents are the main pathologies being defined. Interestingly, some of these diseases can even cause SDB to develop, when it didn't previously exist.

Two proven examples of the consequences of SBD are post-stroke syndrome and increasing obesity. Studies show that approximately 50% of patients with Congestive Heart Failure have concomitant SDB. In many cases it is not clear which comes first, although it is recognized that a snowballing effect occurs between SDB and these other diseases.

It is generally recognized that approximately 20 million Americans have undiagnosed SDB. Due to the significant increase in obesity and allergies occurring not only in the U.S.A., but also in many other developed countries, it is expected that untreated SDB is an increasingly serious and costly serious public health issue.

B. Known Treatments for SBD

The current “gold standards” of treatment are the various forms of Positive Airway Pressure devices, Continuous Positive Airway Pressure (CPAP), Bi-level PAP (biPAP) and auto PAP (aCPAP), delivered either by nasal interfaces (nPAP) or oral-nasal masks. In spite of being used since the mid-1980's and the increased sophistication of auto-adjusting units, as well as improved interfaces, all studies show that long-term compliance by patients is very poor, with estimates of 50-70% non-compliance upon diagnosis. It is believed that 20-30% of patients never start using their CPAP device and 50% of those that do, discontinue use within one year. Approximately 50% of compliant patients do not even wear their equipment the entire night, frequently taking it off in the second half of sleep, when more serious SDB events tend to occur. Smart cards documenting the amount of time the PAP is used may only cause the patient to reduce follow-up, if they expect criticism, or the “cold shoulder” from the frustrated therapist

Other modalities of treatment are also frequently rejected by patients, such as Uvulo-Palato Pharyngoplasty (UPPP), Hyoid Arch Elevation (HM), Maxillo-Mandibular Advancement (MMA), Radio-frequency Tongue Base Reduction, Tracheostomy, and oral devices. Tonsillectomy and/or Adenoidectomy can be an appropriate interventions. However, these treatments comprise costly surgical procedures, wherein the risk of serious complications is greater when the procedure is performed in adulthood. Attending to allergies, general health conditions and life style can decrease tonsillar and adenoidal enlargement, but this requires time and resources that are frequently deficient. It also requires a motivated patient.

Success rates for UPPP are 50% or less and even those patients can still have some O₂ desaturations and respiratory effort related arousals (RERAs), and complications from the surgery. HM, MMA and tongue base reductions can be up to 90% successful, but more than one procedure may be required, are costly. Further, the risks of complications cause these surgical treatments to be accepted only by a small percentage of patients. Tracheostomy is obviously 100% effective, but it too, can also have some negative consequences and usually is done as a last resort and frequently is rejected.

The awareness of SDB and its serious nature is exploding in the medical community and the general public. Multiple high tech devices for diagnosis are being deployed, sleep lab capacity is being overloaded, and even home diagnosis and home diagnosis services are multiplying rapidly.

This revolutionary increase in awareness and diagnosis necessitates a newer as well as a more acceptable approach to treatment. As 50-70% of patients do not comply with PAP therapy and few accept other modalities, a large portion of SBD patients have no effective treatment. Thus the most common therapy now is little or no treatment. This lack of treatment is not acceptable in modem medicine.

One object of the present invention and its mode of implementation is to offer another alternative to CPAP and other current treatments that aim to achieve a higher compliance rate and an active participation of the patient for compliance with other health promoting issues. At minimum, the present invention provides oxygen saturation preserving treatment for already diagnosed patients who have given up on CPAP, or refuse to start.

It is hoped that patients using this new device may prevent the consequences of recurrent hypoxia and continue follow-up with their sleep physician. It is alos an object of the present invention to provide a device that will help to recruit the patient into an interactive health rebuilding, who takes a proactive role, and in enhancing their own health. All three of these are extremely valuable tools, with long term benefits.

C. Prior Art Devices and Treatments Using Sensor Alarms and/or Aversive Stimuli

The use of sensor inputs, alarms and aversive stimuli for therapeutic conditioning goes back almost thirty years, with the development of infant apnea monitors and alarms, bed wetting alarms, snoring treatment devices and self-destructive behavior de-conditioning devices. In 1969, Crossley, in U.S. Pat. No. 3,480,011 disclosed a snore-detecting collar and shocker for snoring reconditioning which he believed to be effective if used. In a technologically related patent, Crossley, in U.S. Pat. No. 4,715,367 and then added to this art the capacity to also treat bruxism and sleep apnea in 1987. The main problems with electrical stimulation to the very sensitive anterior neck region are the patient's aversion to it, which would likely result in a lack of patient compliance with such a testing regime. Further unacceptable EEG arousals caused by the aversive stimulation could worsen excessive daytime sleepiness (EDS).

Wall, U.S. Pat. No. 3,696,377 uses a simple system with a microphone, tape or cassette player and a small earphone to decondition snoring. One circumstance not accounted for in Wall's system was a snoring bed partner, and the lack of aversive electrical stimuli severely reduces the response to conditioning.

Mooza discloses a device in U.S. Pat. No. 3,834,379 that employs an aversive electrical stimulator on the biceps to decondition self-destructive blows to the head or face. Fischell, et al., U.S. Pat. No. 4,440,160 added to this technology by removing the helmet required by the Mooza device, and by replacing the helmet with a narrow headband.

McVaugh, U.S. Pat. No. 3,998,209 discloses a snoring treatment system employing four negative reinforcers including flashing lights, pillow buzzer, armband vibrator and electric shocker; and two positive reinforcers that include verbal rewards and an M&M dispenser. McVaugh's negative stimuli could be delivered singly or in fixed combinations. The positive reinforcers required waking up to turn off the negative stimuli by pressing a button for 15 seconds. This working requirement would result in a very significant arousal. However, the electric shocks also helped decondition the patients, and McVaugh claims that the patients rarely received their M&Ms or positive verbal reinforcers, as snoring was terminated early.

Rosen, et al., U.S. Pat. No. 4,220,142 utilizes a mike for snore and bruxism detection, and an auto-adjusting alarm responding to a counter keeping track of events, and memory capacities to display the number of events for the patient to review on the device. Later an actimeter was added, that temporarily disabled the alarm and counter when the patient got out of bed, and for a brief period when they returned to bed.

Except for the infant and children's apnea alarms which required a nurse or parent to arouse or move the patient to abort an event, no active medical intervention other than T&As, intubation or tracheostomy was available to treat obstructive sleep apnea or other SDB syndromes until the 1980's when CPAP was developed in Australia (See, Sullivan, Issa and Berthon-Jones, et al.) Reversal of obstructive sleep apnoea by continuous positive airway pressure delivered through the nares”, Lancet 1981; 1:862. This took almost a decade since the syndrome was being first reported (See, Guilleminault, Dement. “Insomniac and Sleep Apnea: A New Syndrome”, Science 1973; 181:856-858).

Since then, technological improvements in device and interface design have brought about some increase in compliance. However, all forms of PAP therapy merely provide a temporary pneumatic splint by helping to keep the patient's more collapsible upper airway patent by blowing in pressurized air. No truly significant advancements have been made, aside from improvements in adjusting the inhalation and exhalation pressures, better fitting masks, the capacity to document compliance and events, and even allowing for PAP devices to have diagnostic capacities and be computer and Internet e-medicine enabled. These various additions, that are increasingly expensive, are well known to those familiar with the art, and all competing with each other. Nonetheless, over-all patient compliance is still very unsatisfactory. See, U.S. Published patent application Ser. No. 0020124848 by Sullivan and Lynch; Berthon-Jones, U.S. Pat. No. 6,138,675; and Sun, Crouch, et al., U.S. Published patent application Ser. No. 20020022973.

Two more recently developed, accurate home diagnosis devices have also been deployed (Somte by Compumedics, and WATCH-PAT 100 by Itamar & Respironics), adding to the growing list of screening devices, and services.

All CPAP devices however are still very temporary “velo-pharyngeoplasties” that collapse when turned off and not complied with. As such, room for improvement exists. The present invention and method of implementation seeks such an improvement by attempting to bring about a longer lasting “velo-pharyngeal stenting” process, by increasing the resting and sleeping airway patency, responsiveness and CNS global control, through decreasing the many factors, including the patient's health, that lead to narrowing and collapse.

Before presenting other attempts at SDB treatment devices, it should be noted that the use of interferential pulse stimulators, to induce muscle contractions, goes back to the 1960's, and were used by Russian and Soviet Block athletes for muscle strengthening purposes. This type of stimulator uses two high frequency currents, one in each electrode and differing slightly from each other (e.g., 2000 Hz & 2150 Hz, or 4000 Hz & 4175 Hz). These currents pass through the skin painlessly, with low impedance, penetrating to deeper tissue layers. Then, intersecting at the deeper muscle level, an inferential stimulation pulse occurs from the difference between the two higher frequencies (150 Hz or 175 Hz, in this example) that affects the deeper tissue. This use of inferential currents allows stronger muscle contractions to be brought about without large, painful and disturbing currents being required at the cutaneous level, and are especially useful in very obese patients. Larger, non-inferential currents would also use up battery power more quickly, in a portable, patient worn device.

In 1985, the use of a physiological laryngeal pacemaker was tried unsuccessfully (Kaneko. et al, TransAm Soc Artif Intern Organs), and then attempts were made at phrenic nerve stimulators for diaphragmatic pacing, also with disappointing results. (See Mugica, et al, PACE Vol. 10). (Attempts at cardiac pacing for SBD are currently under investigation).

Meer, U.S. Pat. No. 4,830,008 introduced implantable stimulators for therapy, with a CPU module that could distinguish between central and obstructive apnea, thus providing different timing to treat each. However, these trials also were not clinically useful in medical management for SDB.

The next attempt at a device to detect and intervene in SDB with stimuli was introduced by Timme, in 1987, primarily for use in infants and young children with sleep apnea (See, U.S. Pat. No. 4,694,839). The apnea monitor would trigger a foot vibrator and a neck stimulator. Although a neck-tapping device was used, electrical energy or pressurized air was also considered. The stated purpose was to end the event through arousal, but no significant human-like interventions were incorporated, or envisioned. Unfortunately, the stimuli used by Timme may have not been very effective as the stimuli led to significant arousals (which should be avoided) while inadequately treating the SBP. Reducing respiratory effort related arousals (RERAs), whether self induced by obstructions, hypopneas, central apneas, or caused by PAP equipment, is considered essential in SDB treatment. See Philip, Stoohs and Guilleminault, “Sleep fragmentation . . . ” and its references in SLEEP 17(3): 242-247 from 1994. The affect of arousals should be factored into, and dealt with. Arousals by any treatment system, and the means to reduce them should be incorporated within the device and its deployment.

In 1992, Shannon and Bowman, U.S. Pat. No. 5,123,425 disclosed a different neck stimulator collar that could detect and treat apnea. Transcutaneous electrical pulses were again employed to contract the genioglossus and other muscles affecting upper airway patency. In a related 1993 patent, U.S. Pat. No. 5,265,624 there was disclosed, the addition of wireless stimulation into the mouth, generated from the neck collar. It is believed that a drawback of this system was the disturbing arousals caused by this system, which likely reduces the rate of patient compliance with the system.

In addition, transcutaneous submental stimulation was investigated as a therapy (See Miki, et. al., “Effects of electrical stimulation during sleep on upper airway in patients with OSA” in Am Rev Resp Dis 1989; 140:1285-1289, and Edmunds, et. al., Am Rev Resp Dis 1992; 146:1030-1036).

Taylor, U.S. Pat. No. 5,458,105 discloses a different anti-snore apparatus, marketed by The Sharper Image Corp. Of San Francisco, Calif. The Taylor device utilizes a vibrator attached to the wrist and a snore-detecting microphone. One drawback is that a snoring bedmate would also possibly set off the stimulations.

A relatively new device is disclosed in Francis and Loeb, U.S. Pat. No. 6,240,316 and U.S. patent application No. A1 2001/0010010, which is believed to be being tested by Advanced Bionics Corp., the leading maker of cochlear implants.

The Francis and Loeb device uses implantable throat stimulators, utilizing their BION devices (bionic neuron technology), which can be wirelessly powered and controlled from outside through inductive or RF coupling. These small stimulators are 0.3 to 0.4 cm and 1 to 1.2 cm and can be implanted by a large hypodermic needle. The coordination of stims is attempted by an entraining technique matched to an airflow sensor held over the nose and mouth. Although this approach offers hope in the future, implantations of any kind and the complex coordinations that may be required all could present unforeseen difficulties.

The most recent cervical stimulator was by Mechlenburg and Gaumond in published U.S. patent application No. A1 2001/0018547 and was refined and tested by Respironics. It a magnetic stimulator collar, intended to focus its stimulations into the throat, contracting some of the muscles that dilate the upper airway, such as the genioglossus and various hyoid arch muscles. The device appears to employ rather large and frequent stimulations, in patients with high Apnea-Hypopnea Indexes (AHI) that has the drawback of possibly causing excessive heat. Self-adjusting of stimulations based on feedback, timing of events and responses is embodied in their design, along with possible wireless control from a unit separate from the patient. A problem that can be encountered with this device may be inadvertent vagal nerve stimulation and difficulty with coordinations from inappropriate or contradictory hypoglossal and recurrent laryngeal nerve stimulation. Another potential drawback is that many patients would be resistant wearing a collar anyway, for multiple reasons.

Direct stimulation of the hypoglossal nerve with implanted stimulators has been tried, and can effectively increase airway patency (See Eisele, et al “Direct hypoglossal nerve stimulation in obstructive sleep apnea”, Arch Otolaryngol Head Neck Surg 1997; 123:57-61). Due to the potential surgical problems, cost, and the fact that only one side could be used, this will not be a solution to the crisis of non-compliance with CPAP.

Attempts are currently being made to treat SDB by indirectly stimulating the hypoglossal and recurrent laryngeal nerves with non-invasive vestibular stimulators. See, Mecklenburg & Lattner, U.S. Pat. No. 4,827,935 and published U.S. patent application No. 0020072781. Various adjustment algorithms are entailed and an attitude sensor, but this approach does not include diagnostic capacities. Bilateral stimulation is described to counteract any unpleasant effects from semi-circular canal reactions. Inducing a rocking sensation to help the patient go to sleep is also described. Of course, it is not known if there would be any negative affects from chronic stimulation to these nerves, and it cannot produce or enhance the complex Global CNS and peripheral control required to produce healthy breathing. Unlike the present invention, this device suffers the drawback of not recruiting the patient into a process of taking more responsibility for improving their health.

Many studies have indicated that significant weight loss, and proper attention to treating upper respiratory allergies can improve SDB symptoms. Not sleeping on one's back, in the supine position, is also very helpful. This could perhaps bring 50% improvement in many patients, and is basically a costless therapy.

Interestingly, it has been shown that patients with sleep apnea already make some “natural” compensations on their own, to maintain upper airway patency in the awake state. See, Mezzanotte, Tangel and White, J. Clin Invest 1992; 89:1571-1579 & Am J Respir Crit Care Med 1996; 153:1880-1887. D. P. White and his colleagues involved in investigating the physiology of the proper functioning of the upper airway and its pathophysiology, presented in this study, proof that awake sleep apnea patients have increased genioglossus EMG tone compared to normals. In Fogel, White, et al., SLEEP 25, 2002 Abstract Sup. #487.J, the authors have shown that during sleep in normals there is not only loss of the “wakefulness stimulus”, but also the negative pressure reflex (NPR), and that genioglossal tone is mainly maintained centrally, by activity in the respiratory pattern generating neurons (RPGN). It is the intention of the current invention to further enhance RPGN activity during sleep and the tonicity, responsiveness and the global coordination, not only of the genioglossus and the geniohyoid, but of all the muscles involved in maintaining upper airway patency, especially the tenso palatini, the palatopharyngeus, the glossopharyngeus, pharyngeal constrictors (middle and inferior}, all the muscles elevating the hyoid arch and those that advance (protrude) the mandible, and to achieve this through a unique and complex interactive, waking and sleeping time reconditioning process. At the same time it should decrease the loss of the NPR and waking vigilance during sleep, producing a “sentry vigilance”, but not increase the tonicity of the levator palatini or superior pharyngeal constrictor (which blocks off the nasal space, to allow proper swallowing) or the muscles that lower the hyoid arch. We cannot do these things for the patient with our current state of the art technology. Perhaps, someday, neural net techniques might be able to accomplish theses complex muscular interactions.

Further art related to adequate SDB treatment involves better understanding noncompliance and its consequences, and indicates how a SDB device that not only treats the patient during sleep, but also recruits the patient's active involvement and treatment into the waking hours could be utilized.

Many studies have indicated that weight loss, attending to upper airway allergies, and adjusting sleeping positions, medication management, etc., can reduce SDB intensity. However, compliance with PAP therapy alone does not actively enhance the patient's desire, or need to make those improvements, or any others. Studies on compliance with PAP indicate personality traits that could be taken advantage of to improve compliance. See, Stepnowski, et. al., SLEEP 25(7): 758-762 & Bardwell, et. al., SLEEP 24(12): 905-909. Stimulating and encouraging active coping and problem solving tendencies could improve overall compliance with other health promoting issues, such as weight, sleep hygiene, etc. To make this new system work best, this is exactly what the person should do.

Patient compliance is especially poor in COPD-OSA overlap syndrome where supporting of the patient's O₂ saturation is even more difficult. Patient's can easily accept sleeping with a nasal cannula, but fight a PAP mask or nasal interface, probably because they are already phobic about breathing adequately and feel more uncomfortable, or “choked off” with any style interface.

Disappointing studies are also now being published indicating that even the latest improved auto-adjusting PAP appliances are not significantly improving compliance See Kendrick, et al, SLEEP (25) 2002 Abstract Sup #030.J.

In looking at the long-term effects of untreated SDB it is apparent that the disorders and their co-morbidities worsen as the patient gets older. See Elmasry, “Sleep Disordered Breathing—Natural Evolution and Metabolism”, Comprehensive Summaries of Uppsala Dissertations, ACTA Universitatis, Upsaliensis Uppsala 2000. As the number of early detections in Yourrg, patients increases, it is expected that the number of non-compliant patients shall increase in the coming years. It is likely that these non-compliant parties will discontinue follow-up with the sleep professional, and will also be unlikely to agree to try PAP therapy again, unless a totally maskless approach is offered.

The CNS damage caused by the recurrent hypoxias of SDB is being increasingly documented, and is of great public health concern as the number of elderly citizens increases. One recent study showed MRI white matter hyperintensities in the brains of SDB sufferers in a study of 41 matched pairs of twins, drawn from the NHLBI twin study and managed through SRI International's Human Sleep Research Program. See Colrain, et al SLEEP (25) 2002 Abstract Sup #004.J. Another very recent study demonstrated significant increased cognitive impairments in patients at a memory clinic that chronically snored and also had cardiovascular disease. See, SLEEP(25) 2002 Abstract Sup #008.J.

Two interesting experiments in conditioning that were reported are important in understanding some of the ways the current invention is intended to work in preventing recurrent hypoxias, and improving over all health, in a progressively less arousal causing manner over a reasonable period of compliance. It has been demonstrated that conditioning done during sleep, passes to the awake state, and that conditioning done in the awake state also transfers to sleep See, Ikeda and Morotomi, SLEEP 19 (1):72-74, & SLEEP 20(11):442-447. The importance of this will be obvious when describing how this new device is to be utilized.

Most apnea-hypopnea events occur in series, or clusters, and most obstructive apneas occur as several expirations progressively lower the functional residual pulmonary reserve. Complete obstruction then ensues at the end of an exhale, with the attempt to take the next breath, when the negative pressure required to collapse the airway is at a minimum. See, Morelli, Arabi, et al, Am J Resp Crit Care Med 155:155A419, 1997. That particular apnea ends with an arousal. However, before oxygen reserves can be replenished after that apnea event, the next apnea event or severe hypopnea event occurs. The worst desaturations occur at he end of these cluster. Therefore stopping the series of clusters at the beginning would be most beneficial. A long period of quiescence may then ensue, especially in mild and moderate cases of SDB.

One object of the present invention is to provide a device that is quiescent during these times and that is not a hindrance to sleep, in contrast to CPAP devices and their interfaces, that operate during both apnea events and quiescence events.

Many of these milder patients either refuse, or stop complying with PAP therapy very soon after starting, not realizing that they have a progressive, degenerative disorder. Many physicians also may tend to not actually pressure for CPAP therapy in mild cases, merely recommending, allergy care and prevention of supine sleeping, etc. As an analogy, this would be like medically ignoring “mild” pregnancy.

Many of these less severe cases also tend to only develop truly debilitating clusters during the second half of sleep, when REM sleep is more common, which is the very time that up to 50% of CPAP compliant patients remove their equipment.

Although the above described inventions all provide some help in treating sleep disorders, room for improvement exists. In particular, room for improvement exists in providing a device that overcomes the deficiencies associated with the device described above.

Therefore, it is one object of the present invention to provide a device that improves over known devices by being more likely to promote patient compliance than the currently used CPAP device, and more likely to provide effective stimuli to a patient that helps to reduce the patient's apnea related events.

### IV. SUMMARY OF THE INVENTION

In accordance with the present invention, the device is disclosed for treating sleep and breathing disorders of a patient, along with the method of using the device. The device includes a processor for receiving sensor inputs, processing the received sensor inputs, and generating commands through output devices. A first sensor is positionable for receiving breathing sound information emitted from one of the mouth and nose of a patient. The second sensor is positionable on a patient for receiving breathing sounds information from a patient's chest cavity. A third sensor is positionable for receiving information relating to the amount of chest expansion of a patient. A first output device is provided that is capable of providing an auditory signal to a patient. A second output device is capable of providing an electrical signal to a muscle group of a patient that simulates a human touching event. The first, second and third sensors, and the first and second output devices are operatively coupled to the processor to permit the processor to receive information input from the sensors, process the input information to the detect the existence of a sleep-breathing disorder event, and to generate command to at least one of the first and second output devices. The command is capable of directing the at least one output device to provide a series of progressively intrusive stimuli designed to condition the patient to terminate the sleep breathing disorder event, and ultimately, return to a more normal sleep pattern.

One feature of the present invention is that it comprises the interactive, rehabilitative robotic device for the holistic treatment of a wide variety of sleep disordered breathing syndromes, including simple snoring. The device is capable of functioning as two, separate devices, useable for separate purposes. The device employs both operant and respondent de-conditioning and re-conditioning processes, and motivational processes to treat sleep disorder.

This feature has the advantage of helping to cure and treat sleep breathing disorders. During sleep, the device has the potential to stop or at least reduce sleep disordered breathing events, and helps to de-condition the patient against a large number of factors that lead to, or cause, breathing obstructions and breathing cessation, while helping to simultaneously recondition global CNS and peripheral coordination or breathing.

During waking hours, the system can be switched to become a training device, to be used by the SDB patient to strengthen all of the neurological and muscular coordinations required for normal breathing, by enabling the user to better practice and reinforce a specific “reflexic exercise” that increases or helps maintain upper airway patency. During such practices, the patients are better able to condition themselves to more quickly respond, in the same reflective fashion, to the various intersessions the device uses during sleep treatment when a sleep disordered breathing event is detected. This feature has the advantage of helping to reduce arousals of a patient from a sleeping state caused by stronger intersessions provided by the device.

A second primary feature of the present invention is that it is capable of providing more humanoid-like intervention to the patient. It is believed that these humanoid-like interventions will be ultimately more successful in treating a patient's sleep disorder, and will be better received by the patient than non-humanoid-like interventions of the type practiced by some of the prior art.

As part of the humanoid-like intervention, the system uses the patient's name intermittently as part of its verbal commands, and provides verbal instructions that are specific to the type for breathing fault and positioning of the patient. The device includes a provision to employ either interchangeable, or programmable language chips, so the device can be employed and programmed to issue these verbal commands in the particular native language of the patient.

Additionally, the system can include a voice chip that can be programmed to contain motivational statements or capable of being “whispered” to the patient, to provide almost subliminal motivation to the patient while sleeping, if the patient elects to enable that particular function.

Another aspect of the device is that it includes a sensor and microprocessor that are capable of distinguishing between snoring, more significant air flow reduction of the type that is likely to result in hypopnea, total obstuction, the hypoventilation phase of Cheyne Stokes respiration and central apnea, and then is capable of adjusting its verbal commands to the particular event. This feature has the advantage of enabling the verbal commands to be tailored in kind, and degree, to the specific breathing disordered event that is then occurring, to more effectively treat this disordered event.

Another example of this humanoid intervention, is that the system has the capability of inducing electronic current into the patient that simulates the patient's shoulder being shaken either gently or intensely by a bed partner or medical professional. This shoulder shaking is employed to help promote proper breathing and termination of sleep disordered breathing events, in a manner similar to which a gentle poke to a snoring husband's shoulder by an awakened wife will often cause the husband to cease his snoring and return to a more normal, and more importantly, quieter breathing pattern.

Additionally, the system can include the capability to induce an electrical stimulus into the patient's neck to helps to extend the patient's neck, to thus correct an airway that is in an over-flexed, compressing position, and to keep the shoulder in sustained contraction, if so desired, to help correct, on a longer term basis, this over-flexed airway compressing position. This stimulus helps to mimic the humanoid type intervention of the type that the patient might undergo in an effort to help cease his snoring.

Additionally, another humanoid intervention that is the system is capable of flexing a patient's arm intermittently, and to coordinate this arm flexing with shoulder shaking to produce a stronger shaking effect. This stimulus helps to mimic a human-like intervention, as it mimics the intervention of a more aggravated bed partner who is trying to stop his/her partner from snoring by shaking the patient more vigorously, to help bring them out of a snoring phase, and cause them to go into a more quiescent sleeping phase.

Another humanoid-type intervention is capable of creating is through utilizing interferential currents, from pulse stimulators, to reduce unpleasant cutaneous sensations, and to also utilize aversive “nicking”, stinging or shocking sensations that are typically found in traditional frequency stimulators.

An additional feature of the present invention is that the sensor and stimulator components can be designed to be miniaturized, reliable, and convenient. This feature has the advantage of enabling the device to be made reliable and effective, while reducing the “mass” of the device. As will be appreciated, a smaller device is preferred to a larger device, as a smaller device is less likely to interfere with the user's sleep patterns than a larger device. Additionally, it is likely that the use of a smaller, less intrusive and obstructive device is that it is likely to result in greater patient compliance, when compared to a larger, bulkier, more uncomfortable device, especially if that device is to hooked up, connected to, or worn by the user during the user's sleep. To help reduce production costs, standard sensors and stimulators can be used, along with micro-electro mechanical system technology, that can be programmed and designed to work with the present invention. The components can be made small enough to be used on very young children, yet powerful enough to be used on very large, or obese adults.

Another feature of the present invention is that a micro-processor is provided that performs a variety of functions for the device and process, including digitally analyzing and storing data relating to the speed, quality and duration of responses to various stimuli. The processor can be programmed to employ artificial intelligence to help re-configure itself to increase or decrease or change the manner in which the various stimuli are delivered in response to various breathing defects in order to enable the device to function more effectively for each patient.

The digital processing system of the present invention can include a digital voice recorder having a play-back feature, that is capable of recording and playing the name of the user, so that the patient's name can be employed as a part of the stimuli given to him. Preferably, the digital voice recorder is erasable, and re-recordable, so that a single voice chip can be used for a variety of patients, but reprogramming it both with the new patient's name, and also with the new patient's stimuli and treatment regime. The smart card can be used as a nonvolatile, re-recordable memory to serve this purpose.

It is also a feature of the present invention that the primary module of the device includes a display screen that is coupled to a touch pad or other inputing device. The display screen can be used by the patient and/or medical practioner to facilitate the process of inputing information into the device.

More importantly, the display screen can be used as a device for displaying reports to users and their medical practioners relating to patient's sleep disorder. Example of such reports could include the patient's oxygen saturation during a sleep event, breathing pattern, snoring levels, stimuli given to the patient, responses to stimuli, and other information relating to the occurrences and quality of the patient's sleep experience, and the patient's response to any treatments given during his sleep experience.

These and other features of the present invention will become apparent to those skilled in the art upon a review of the following drawings and the Description of the Invention, which are perceived by the Applicant to be the best mode of practicing the invention now known to the Applicant.

### V. BRIEF DESCRIPTION OF THE DRAWINGS

FIG. 1 a is a frontal, somewhat schematic view showing a patient wearing the sleep disordered breathing treatment device of the present invention;

FIG. 1b is a rear-view of a patient wearing the sleeping device;

FIG. 2 is a diagramatic view of the various components of the present invention;

FIG. 3 is a diagramatic view of the various components of the present invention and their relation to the central micro-processor of the unit;

FIG. 4a is a flow chart of the first exemplary stimulus pattern or “CAST” useful in the present invention;

FIG. 4b is a flow chart of a second exemplary stimulus or “CAST” progression pattern;

FIG. 4c is a similar, flow chart view of a third “CAST” or stimulus pattern useable with the present invention;

FIG. 5a is side elevational view of the arm module of the present invention;

FIG. 5b is bottom view of the arm module of the present invention;

FIG. 5c is a top view of the arm module of the present invention;

FIG. 6 is a sectional view of the arm module of the present invention, showing the present invention as wrapped around the upper arm of a patient;

FIG. 7 is a somewhat schematic, side elevational view of an ear-piece module of the present invention, showing various accessories and modules that can be attached thereto;

FIG. 8 is front, somewhat schematic view of a main module of the present invention;

FIG. 9 is an anitomical view of the musculature of the rear chest area of a human being;

FIG. 10 is a schematic view of the positioning of the supraspinatus muscle, showing its relative position in the human body;

FIG. 11 is a schematic view of a human skeleton showing the position and shape of the levatorscapulae muscle of a human being; and

FIG. 12 is schematic view of a human skelton showing the position of the trapezius muscle.

### VI. DETAILED DESCRIPTION

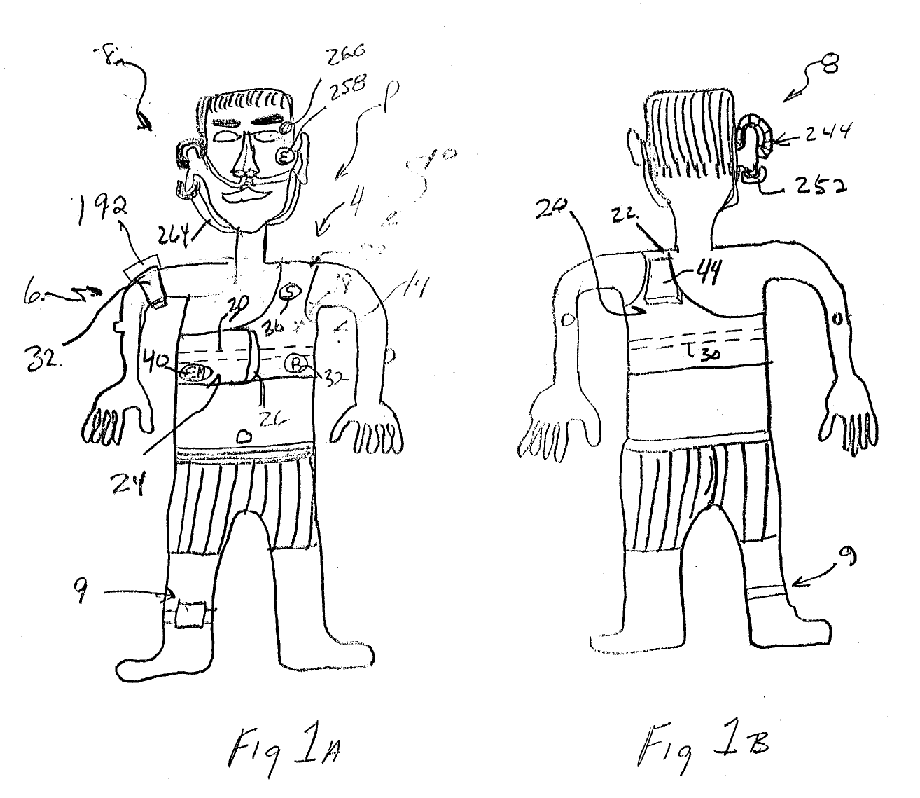

As best shown in FIGS. 1a and 1b, the sleep disordered breathing (SDB) treatment device 10 of the present invention includes a chest module 4 that is placeable around the chest of the patient and an arm module 6, that is placeable around the arm of the patient, adjacent to the patient's biceps and triceps muscles, and a head module 8 that is worn over the head of the patient and which is coupled to the patient's ear. A leg-mounted actimeter 9 can be placed on the leg of the patient to monitor leg movement of the patient, and also to induce a stimuli to cause a leg movement response in the patient that has been found by the Applicant to arouse the patient out of a sleep disordered breathing (SDB) event, and to help resume normal breathing.

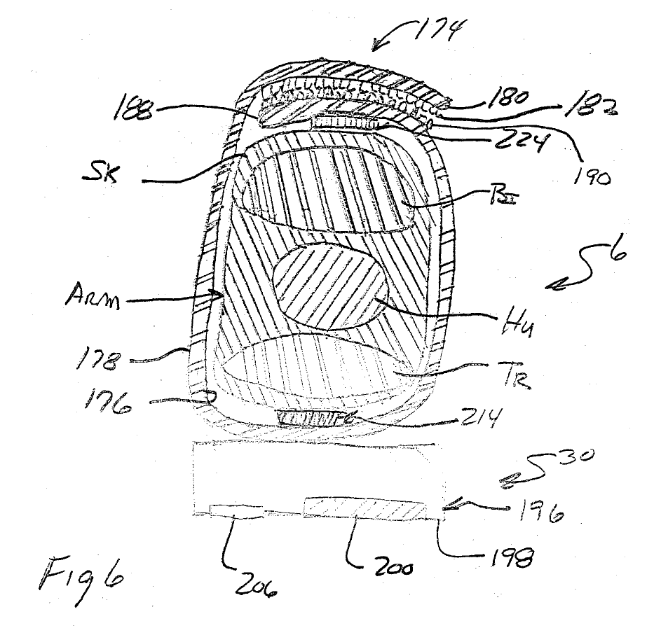

The chest module 12 is best shown in FIGS. 1a, 1b and 8, as including a chest-engaging shoulder wrap 14 that may be similar in configuration and construction to SHARPER IMAGE® magnetic therapy shoulder wrap that is distributed by The Sharper image Corporation of San Francisco, Calif. Unlike the Sharper Image shoulder wrap, there is no need, in the present invention for the magnets and hot and cold jell inserts that accompany the SHARPER IMAGE® magnetic therapy shoulder wrap.

The shoulder wrap 14 includes a chest-engaging portion 18 to engage the chest of the user, a back engaging portion 20 that is disposed adjacent to the scapular region of the back of the patient, and a shoulder engaging portion 22 that extends over the shoulder of the patient. An adjustable strap member extends between chest engaging portion 18 and the back engaging portion 20 to wrap around the user's chest. Each end of the adjustable strap member may include one half of a hook and eye fastening (e.g. Velcro®, member, that enables one end of the adjustable strap member 24 to be engaged to the back engaging portion, and the opposite end of the adjustable strap member 24 to be engaged to the chest engaging portion 18 of the chest engaging shoulder wrap 14 to secure the wrap on to the patient's chest. A hook and eye fastening material 26 is disposed at the end of the adjustment strap 24.

A plurality of sensors are fixedly coupled to the chest engaging shoulder wrap 14. The sensors can be permanently affixed to the shoulder wrap 14, or alternately can be removeably coupled, such as through a snap member, to permit the sensors to be replaced when necessary. The sensors include a chest expansion sensor 30, that essentially comprises a tubular member that extends around the chest, and is utilized to measure the expansion of the chest, which provides an indication of the breathing, (or lack thereof) being performed by the patient.

A breathing sensor 32 is positioned adjacent to the chest cavity of the patient, and functions similarly to a stethoscope, insofar as it senses breathing sounds. A snoring sensor 36 is positioned on the shoulder portion 22, to detect snoring sounds and breathing sounds emitted from the mouth and nose of the patient. Snoring sensor 36 essentially comprises a microphone. An EMG sensor 40 may be placed upon the chest or strap portion.

Each of the sensors described above, including chest sensor 30, snoring sensor 36, breathing sensor 32 and EMG sensor 40 are provided with either a hard wire or wireless communication, to provide input to the main module 44 of the device 10. The main module 44 is disposed on the back of the patient, and is best shown in FIG. 8.

The main module 44 includes a case 50 having a base portion 54 and flip top lid 56 that is hingedly connected to the base portion 54 by a hinge arrangement 58, that can comprise a conventional piano-type hinge, as shown in the drawings, or alternately, a less expensive living hinge. The case 50 houses the electronic circuitry that comprises the “brains” of the device 10, the most component of which is the processor.

Additional componentry can include a voice chip, a non-volatile memory, such as a smart card, and various cableing and wires to provide communication, along with a radio frequency transmitter and receiver for wireless communication. A power source, such as a plurality of batteries provide the power for the main module 44 to operate.

The main module 44 contains a LCD display screen 62, that may be similar to an LCD display screens that one might find on a PDA or cell phone. The LCD display screen 62 is large enough to include a multi-character, and preferably, multi-line message. The LCD display screen 62 may be similar to a PDA, insofar as the screen may be a touch screen so that the screen 62 also serves as an input device. Alternately, an input device 66 can also be placed on the base of the main module 64. As will be discussed in more detail below, the multi-character message 64 that are placed on the LCD display screen can include things such messages of encouragement (good job), messages regarding the user's weight, along with various menu items (e.g. program outputs, display results) that permit the user to navigate through the operation of the device.

In main modules 44 of the type that do not use a touch screen display, an input device 66 can be employed, such as an input device 66 that includes a 12-button touch pad 68, that is similar to the pad used on a cellular phone, that permits the user to input information into the main module 24. A joystick control 70 can be provided for a facilitating navigation among the various menu items that are shown on the display. Additionally, additional operation button, such as a power button 74 and OK/enter button 80, and clear button 82 can be disposed on the face, and employed by the user to operate the main module 44, and hence the device.

The main module 44 should also include an external speaker 84 on its base for providing an auditory message to the user that is either an addition to, or in lieu of the ear phone device that will be described below. A microphone 88 is provide for enabling the user to record material onto the voice chip contained within the device, such as the user's name, words of encouragement, semi subliminal messages and the like. Additionally, the microphone 88 can be employed to receive voice commands, if the main module 44 is designed to respond to voice commands.

An internal, transmitter/receiver/antenna 92 is provided for enabling the main module 44 to communicate wirelessly with the various sensors and output devices of the present invention. A plurality of jacks and/or plug receivers and/or ports (collectively referred to herein as “ports”) 96 are provided on the main module 94 for enabling the main module 44 to be hard-wired to various sensor-type input devices and/or output devices.

Among the devices to which can be hard-wired to the main module 44 are first stimulator member 100 and a second stimulator member 116. The first stimulator member includes a cable 102, and a plug 104 that is coupled to jack 96 of the main module 44, and skin engaging pad member 106. A larger surface area electrode 108 and a smaller surface area electrode 110 are disposed on the pad member 106 and are positionable, through adhesive area 112 in contact with the skin of the user and provides an appropriate stimulus.

Similarly, second stimulator member 115 includes a cable 118, a plug 120 and a pad 122. Disposed on pad 122 is a larger surface area electrode 126 and a small surface area electrode 128. An adhesive area 130 is provided for adhesively coupling the pad 122 to a skin surface of the patient. As will be discussed in more detail below, the smaller and larger surface area electrodes comprise a dual-type electrode that can provide different types of stimulation to the area in contact.

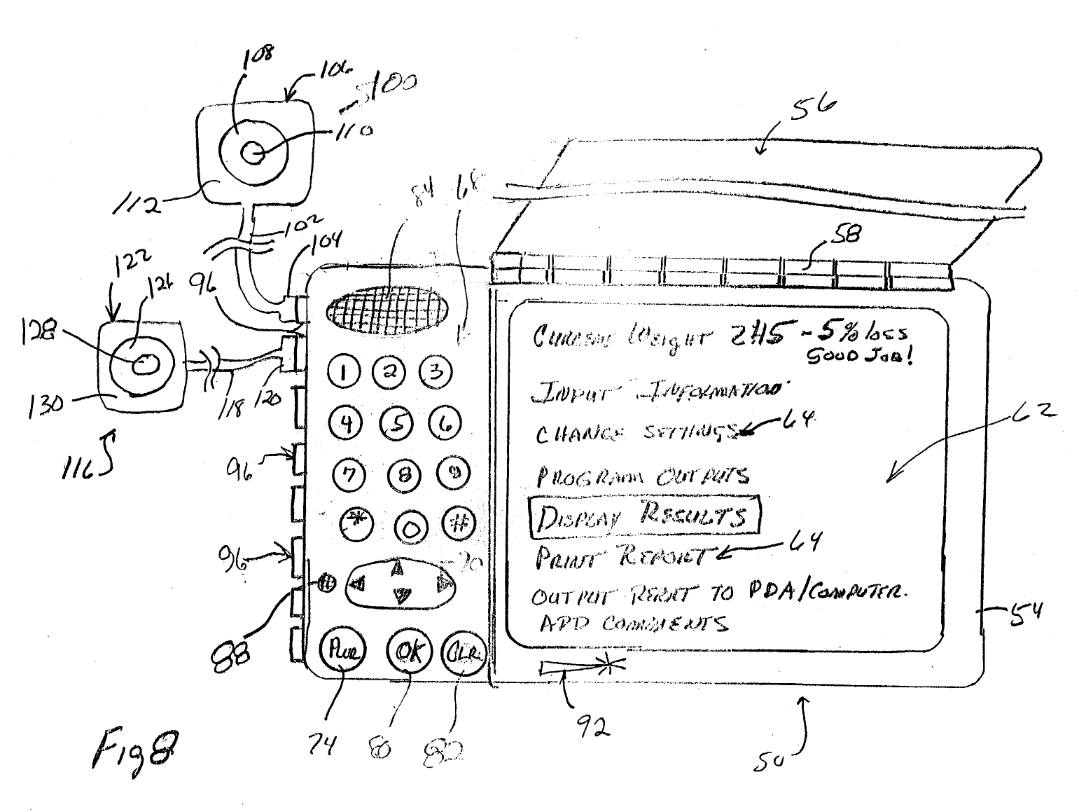

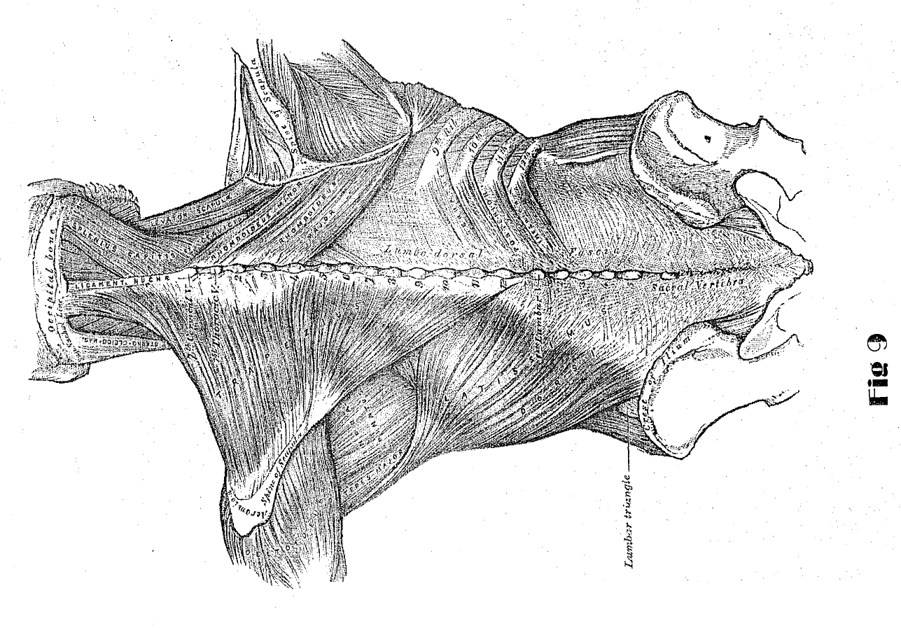

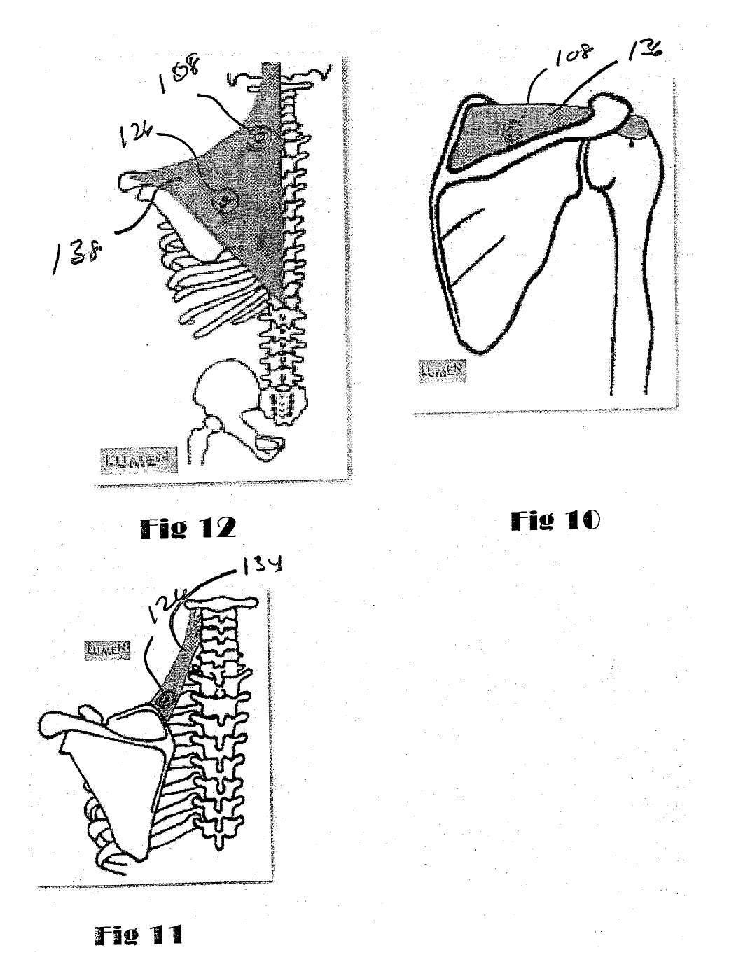

As best shown in FIGS. 10, 11 and 12, the electrodes are placeable on the back shoulder portion of the patient, to induce the stimuli into the levator scapulae, the supraspinatas 136, and/or the trapezius 138. By placing one electrode to give an inferential pulse stimulus onto the levator scapulae, the second electrode gives a appropriate stimulus into the supraspinatas 136, the stimuli can be made to create a sensation in the patient similar to the sensation one receives when ones shoulder is being shaken. Through the introduction of this stimulus, a humanoid like stimulus is provided to the patient, as is discussed in more detail below. Additionally, a stimulus can be induced into the trapezius to either cause shoulder shaking, or alternately, to cause appropriate vibration shocking type stimuli to the patient.

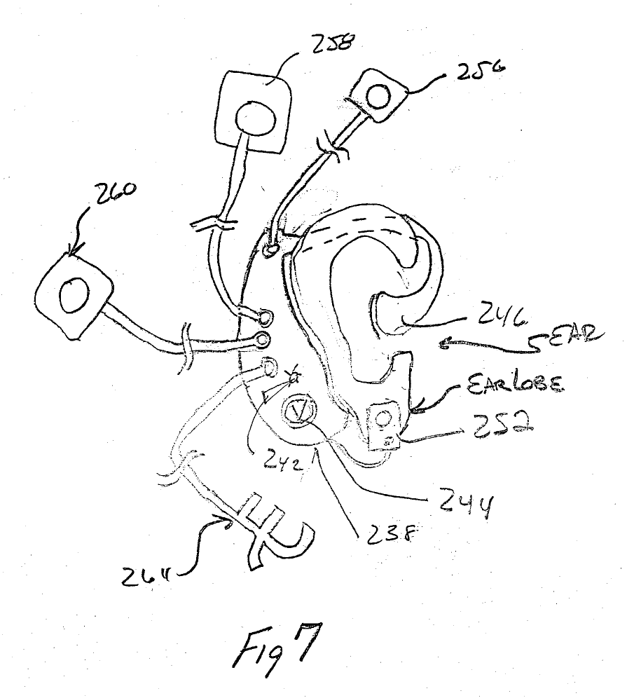

The relation of the various muscles to the remainder of the back of the patient is best shown with respect to FIG. 9 that comprises a copy of an etching from Gray's Anatomy illustrating the various muscles of the back.

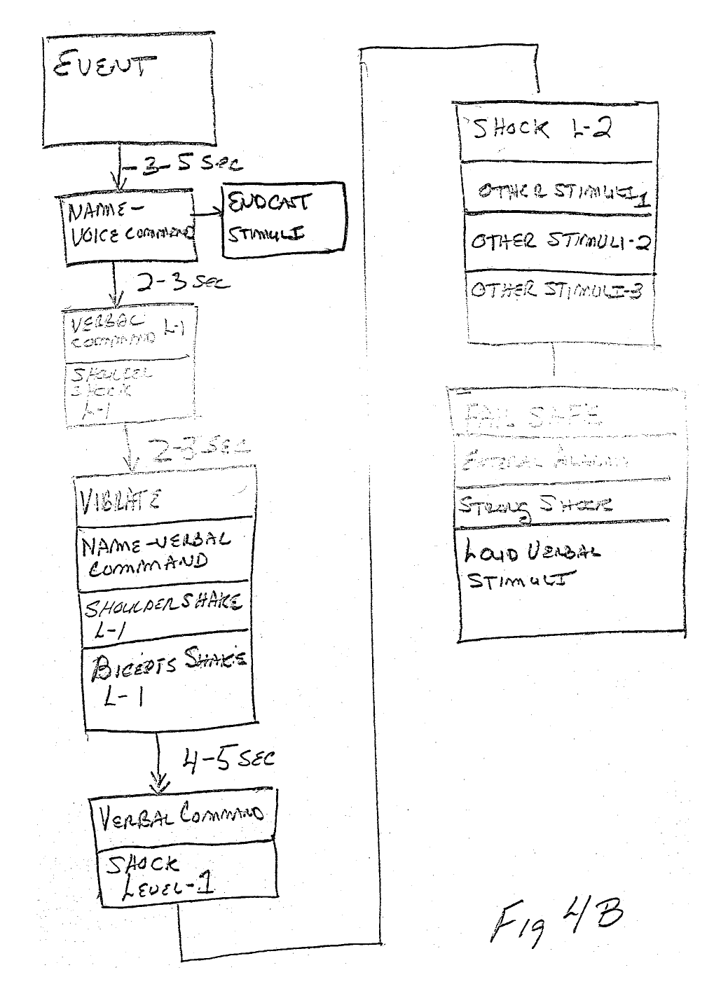

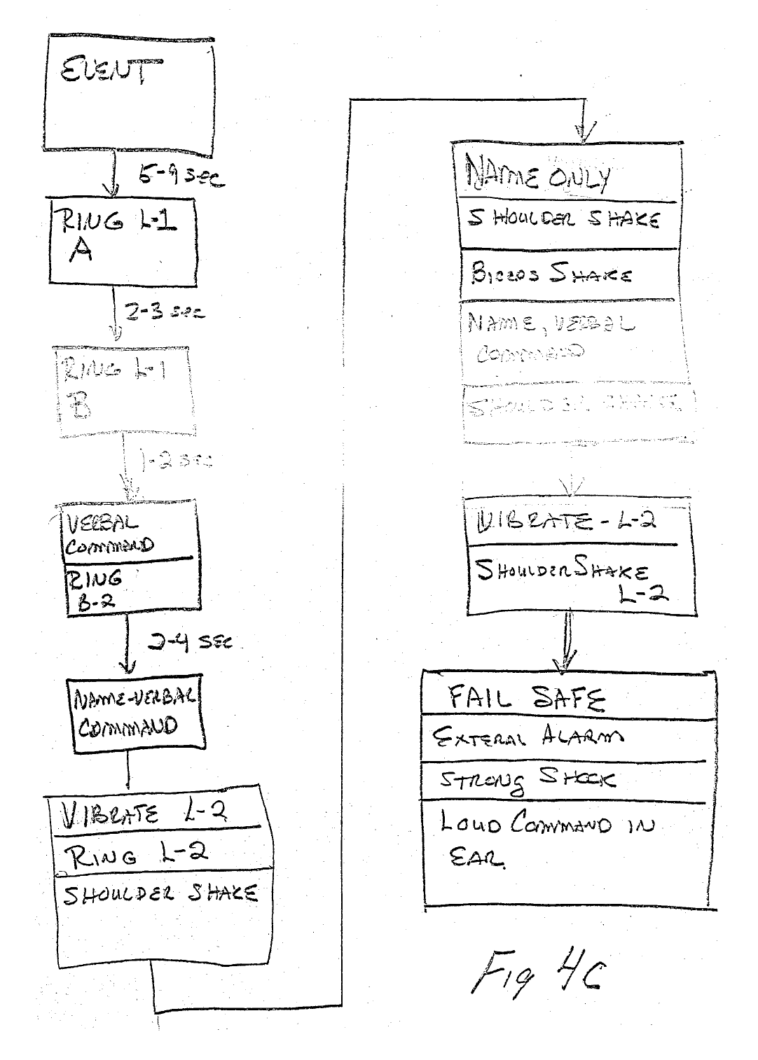

The arm module 6 is best shown in FIGS. 5a, 5b, 5c and 5 and 6 as including a generally rectangular cloth strap 174 having a skin engaging surface 176, and an outer surface 178. The strap 174 includes a first end 180 having the “hook” and portion of a hook and eye fastener material, such as VELCRO brand fastener material that is affixed to the skin engaging surface 176. The strap 174 also includes a second end 188 having a swatch of the “eye” end portion of a hook and eye engaging fastening material, such as VELCRO 190 disposed on the outer surface 178 adjacent to the second end 188.

The arm module processing unit 192 is disposed on the outer surface 178 and includes a case 196 for housing the various electronic components in the arm module processing unit 192. These components can include things such as a transmitter and receiver unit, a small (compared to the main module 44) processing unit for processing commands received from the main module 44, along with various processing units for operating the electrodes and display of the arm module processor unit, along with receiving inputs from the various buttons and control member of the arm module 192. Although it is envisioned that the arm module will receive and transmit information from main module 44 through a radio frequency connection, jacks are provided for providing the hard-wired connection between the main module and the arm module processing unit 192. In this regard, it will be noted that the stimulator members can be hardwired to the various sensors via hard-wired connections 194 and 195.

The case 196 includes an upper surface containing an LCD display 200, that is capable of containing multiple characters and/or graphs 202. A plurality of functions are provided for controlling the operation of the module, including an addition button 206, a subtraction button 208, a function button 210 and an enter button 212. The buttons 206-212 are provided primarily to control the operation of the stimulator electrodes 214, 224 of the arm module 44. The function button 210 allows the user to toggle between various functions, such as time, intensity and the like. The plus or minus function buttons 206, 208 permit the user to increase or decrease the level of the various functions, such as increasing the time of the stimulation or decreasing the time of the stimulation; and the enter button 212 enables the user to lock in a particular level for a function. A power source, such as a set of batteries is included within the arm module processing unit 192 to provide the appropriate operating power for the functioning of the device.

A set of triceps stimulator electrodes 214, and a set of biceps stimulator electrodes 224 are hard-wiredly coupled to the arm module processing unit 192, and are provided for exerting an appropriate stimulation on to the skin surface of the patient and the respective triceps and biceps of the arm of the user to which the arm module is attached. The triceps stimulators 214 include a pair of stimulator electrodes, including a first triceps stimulator electrode 216 and a second triceps stimulator electrode 218. Similarly, the biceps stimulator 224 includes a first biceps stimulator electrode 226, and a second biceps stimulator electrode 228. Similar to the electrode discussed in connection with the main module 44, each of the various electrodes 216, 218, 226, 228 can comprise an electrode having a larger area electrode portion 230, and a smaller area electrode portion 232.

As best shown in FIG. 6, the device is wrapped around an arm, so that the skin engaging surface 176 is placed adjacent to the skin SK of the user on the user's upper arm, so that the strap 174 extends around the arm in a position adjacent to the humerous HU of the user. The triceps stimulator electrodes 214 are placed adjacent to the triceps muscle TI of the user's arm, and the biceps stimulator electrodes 224 are placed adjacent to the biceps muscle BI of the user's arm. The first end 180 and second end 190 are placed in an opposed adjacent relationship so that the swatch of hook material 182 may engage the swatch of eye material 190 of the hook and eye fastener, to keep the strap 174 securely positioned upon the user's arm.

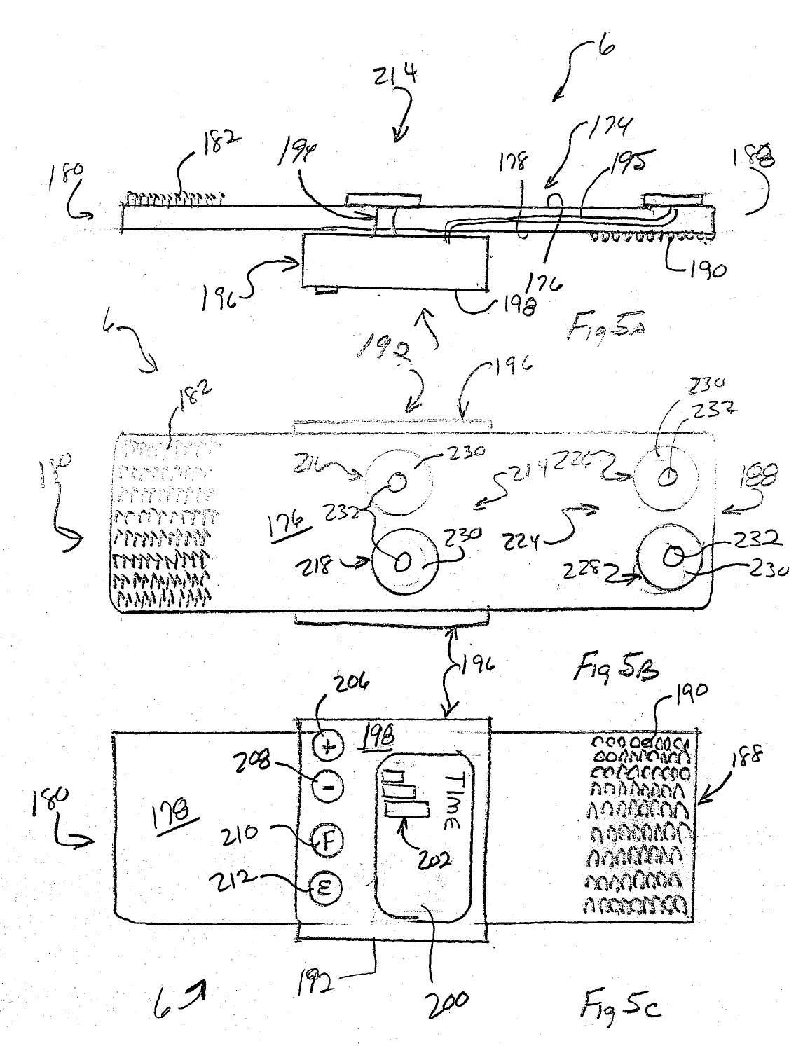

The head module 8 is best shown in FIGS. 1a, 1b, and 7 as including an ear-engagable processing unit 238 that is coupled to the ear of the user. The ear-engaging processing unit includes appropriate circuity for processing the various signals and information necessary to the operation of the head unit 8, such as processing the signals received from various sensors, including the received transmitter member 242 for transmitting these received signals wirelessly to the main module 44. Additionally, the receiver transmitter member 242 is capable of receiving output command signals from the main module 44, which signals are then fed to the various processing circuitry within the ear-engageable processing unit 238, and then delivered to one or more output devices, such as the earphone 246 that is capable of giving an output to the user, such as a noise or voice command, and most importantly, a name containing voice command, that is useful in connection with the method of conditioning the patient of the present invention.

A plurality of “optional” sensors can be coupled to ear-engaging processing 238. The “optional” sensors are not necessary for the operation of the device, but are desireable for the operation of the device, and are provided for sensing various conditions of the patient, of the type that can best be sensed by a sensor placed on the head of the patient.

The sensors that can be coupled to the ear-engaging processing unit 238 include an earlobe oximeter 252, that is provided for sensing the oxygen saturation of the user's blood. An EMG sensor 256 can also be coupled to the ear-engageable processing unit. An EEG sensing electrode 258 is used alone, or in tandem with a combination EOG+EEG sensor 260 can be provided for sensing brain wave information of the user. An EOG sensor is also provided as a part of the combined EOG and EEG sensor.

Finally, a nasal cannula-type sensor 264 can be provided for sensing nasal and mouth emitted breath sounds and/or pressure, or for inducing a flow of air into the patient's nostrils and/or mouth. However, to induce such a flow of air, it is likely that the nasal cannula 264 would be better served by being connected to an air pump or CPAP-type device that is capable of creating a flow of air that can flow through the cannula 264. A volume knob 244 is provided for enabling the user to adjust the volume of sound emitted from the earphone 246, to accommodate patients having different degrees of hearing acuity.

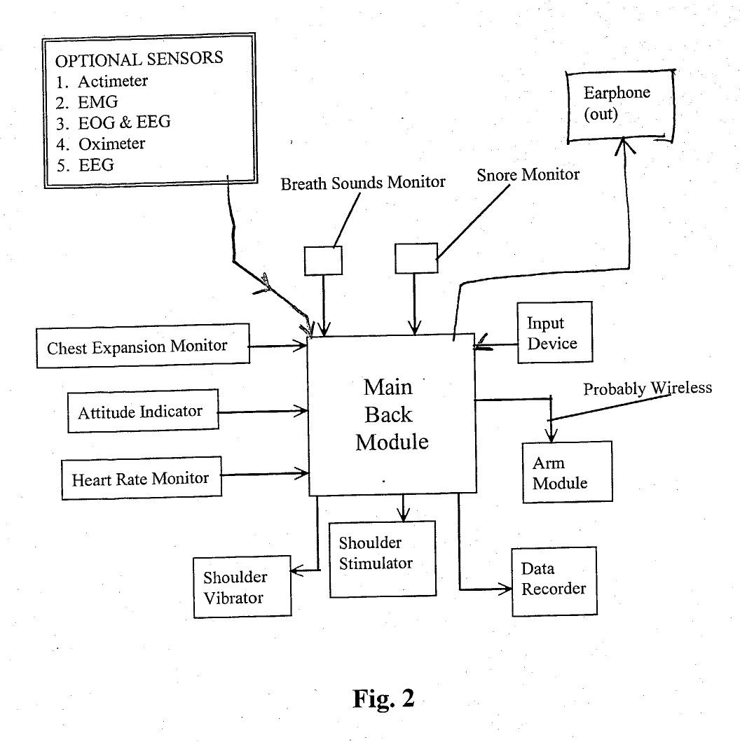

The inter-relation between the various sensor inputs and the main processing module, as contained within the back module will now be described in respect to FIG. 2.

The device includes a plurality of sensors that transmit information into the main back module, that relate to the various conditions of the patient. As discussed above, these monitors include such things as breath sounds monitor, snore monitor, the chest expansion monitor, an attitude indicator, and a heart rate monitor. Additionally, an input device, such as a microphone, and the key pad input device also send signals to the processing unit within the main back module.

In addition to the sensors discussed above, several optional sensors exist, that can provide information about the patient to the main back module, and the processor contained therein. These optional sensors include an actimeter, an EMG sensor, an EOG and EEG sensor and oxymeter, and a second EEG sensor.

The main back module includes a microprocessor for processing the information sensed by the sensor, as directed by the input device, that tells the processor what to do with the sensed information. Upon the detection of a sleep breathing disorder event, outputs are transmitted to one or more of the various output devices that are coupled to the unit. These output devices include the earphone for providing a verbal signal to the patient; a shoulder stimulator electrode that is designed for providing stimuli to the muscles under the shoulder, that can include a shaking type stimuli to provide a humanoid-like stimulus similar to the stimulus one would receive from a spouse shaking the shoulder of a snoring patient, a shoulder vibrator output device, that provides an output to the musculature in the shoulder that provides a vibration type field, and various stimuli to the arm module, both to the triceps and biceps stimulator electrodes.

It is likely that the proximity between the main back module and the shoulder vibrator, and shoulder stimulator will likely cause these to be connected to the main back module through a hard-wired connection. Additionally, breath sounds monitor, snore monitor, heart rate monitor an attitude indicator, and/or chest expansion module, may be designed to be incorporated within the main back module as a part thereof, and as such, would be connected to the main back module for a hard-wired connection.

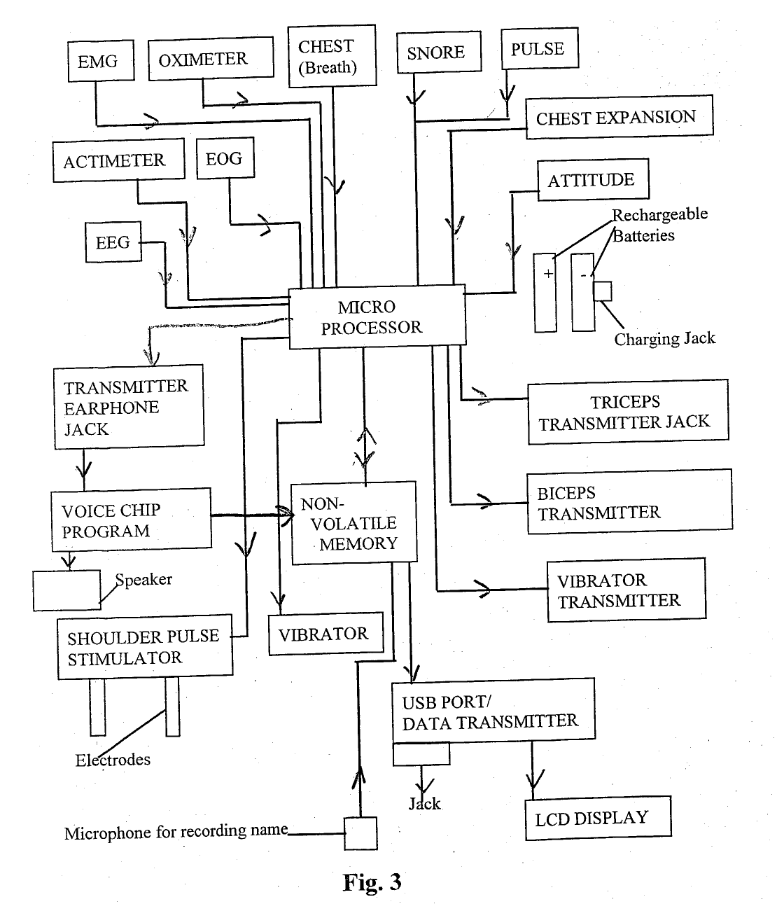

Others of the sensors output devices would likely receive and/or transmit (as appropriate) their signals with the main back module for a wireless connection, due to the fact that they are not proximately located to the main back module. For example, wireless transmitter/receiver within the main back module could be used to transfer data to (or from) the data recorder, the arm module, the earphone and the optional sensors, many of which are located either on the head module unit, or, in the case of the actimeter, are located on the leg. The data recorder could be either hard-wired to the main back module, through a jack connection when data would be downloaded, or alternately, could be wirelessly connected to the main back module, so that date could be reported in real time and displayed in real time at a location spatially removed from the main back module, such as in a room next door from the room in which the patient is sleeping. In FIGS. 2 and 3, the arrows show the directions in which the primary signals are transferred to or from the various components (e.g. the chest expansion monitor), and the main back module.

Turning now to FIG. 3, a schematic representation is shown that illustrates functional relationship of the various components of the device. Essentially, FIG. 3 is an expansion of FIG. 2, and illustrates that the microprocessor that is contained within the shoulder unit, is the “heart and brains” of the device, that is provided for receiving a wide variety of inputs from various sensors, processing the input so received, generating commands, and transmitting those commands to various output devices. As is shown from the arrows, various sensor inputs are received by the microprocessor from the various sensors, and various output commands are delivered to the various output devices.

It should be noted that the microphone for recording a name of FIG. 3 is provided, that can record information on non-volatile memory, the operation of which is controlled by a voice chip program.

The microprocessor and non-volatile memory are in communication with each other, as the non-volatile memory likely includes programming information that tells the microprocessor how to process the data it receives and generate the command it generates. Additionally, the non-volatile memory serves as a repository of data collected by the sensor during the operation of the device. Much like a photograph can be downloaded from a smart card type non-volatile memory contained within a digital camera, a USB port can be provided that contains a jack, to which a USB plug can be attached for downloading data from the non-volatile memory about the condition of a patient during a sleeping episode. This information can include not only the information sensed from the patient, but information about the outputs, such as the shaking outputs, the auditory outputs, and the shocking outputs that are delivered to the patient. The output data can be time correlated with the input data to give the researcher or medical practitioner a chronologically-based report that reflects the user's sleep pattern, including the user's quiescent sleep episodes, and the user's sleep disordered breathing event episodes.

An important component of the present invention is the “CAST”, that comprise the various treatment and stimuli regimes that are rendered to a patient. As will discussed in more detail below, the CAST regimes have two primary purposes. The first primary purpose is to interrupt sleep disordered breathing events to prevent the sleep disordered breathing event from causing harm to the patient, such as by causing the patient to suffer from hypoxia.

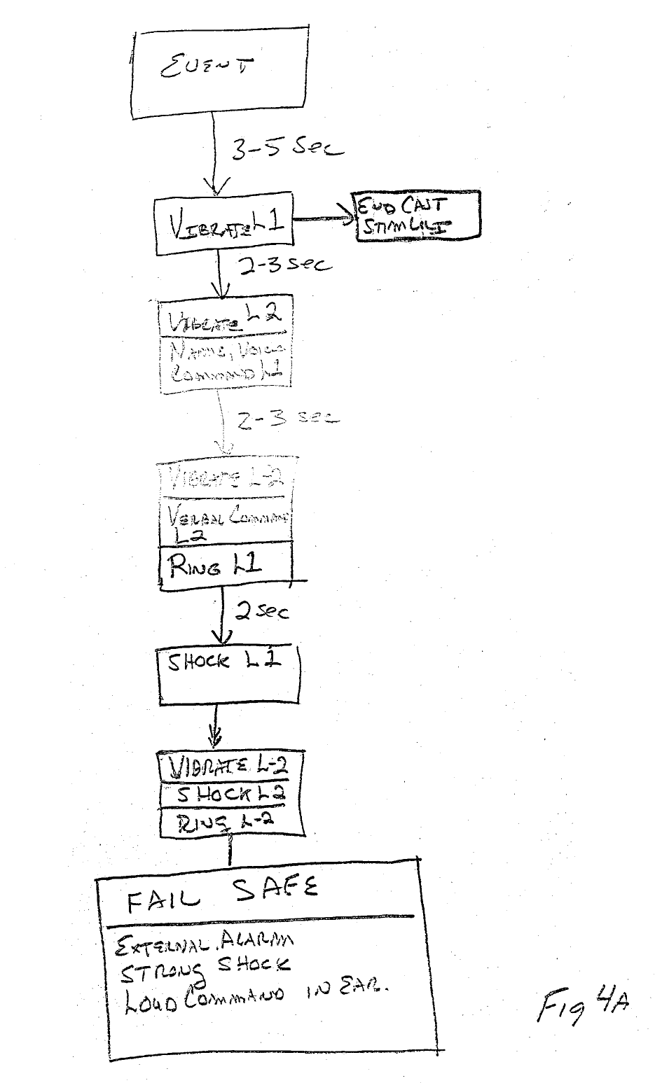

The second primary purpose of the CAST regime is to provide operant and respondent conditioning to the patient to help condition the patient to both decrease the number of sleep disordered breathing events, and to decrease the severity of the sleep disordered breathing events that the user does encounter. Although various methodology behind the selection of the various sleep events within a particular CAST will be discussed in more detail below, three sample CAST's (cue and stimulus training regimes) will be discussed in connection with FIGS. 4a-4c.

Turning first to FIG. 4a, the device will operate in a normally quiescent output rendering mode, until such time as the sensors sense that a SDB event is occurring. The device is designed to insert a 3 to 5 second time lag before providing any stimuli, to give the patient time to self-correct his behavior. The first stimulus that may be given to the patient is a vibration at a low level in the hope that this vibratory stimuli will cause the patient to end his disordered breathing event, and begin breathing normally.

If the vibration is successful, and the breathing disorder is terminated, as determined by the sensors, the CAST stimuli will be ended. As will be appreciated, the CAST stimuli can be ended at any point in the CAST when SDB is terminated. However, for the sake and clarity of the drawings, an “end CAST stimuli” box will not be shown adjacent to each various level.

If the vibratory stimuli is not successful in ending the SDB, a second stimuli of increasing intensity will be rendered to the patient, which in this case, would comprise a secondary level vibration that is coupled with a human voice command such as “Charlie!”, at a first level. If this stimuli is not successful, the patient will be stimulated at a third level that can include second level vibrations, a verbal command (e.g. “stop snoring”), and coupled with a ringing delivered through the earphone.

If this is not successful, after a two second interval, more invasive stimuli, such as a first level shock may be delivered to the patient, which, if not successful will be followed by more invasive stimuli, which may comprise secondary level vibration, a secondary level shock, and a secondary level (louder) ringing within the user's ear. If all of this fails, the fail safe mode of the invention will take over to provide a very strong stimulus to cause the patient to quickly terminate his SDB event. The fail safe stimulus can include a loud external alarm providing a strong auditory stimuli to the patient, a strong shock providing pain to the patient and a loud command delivered to through the earphone into the patient's ear.

A second CAST is shown in FIG. 2b. The CAST is FIG. 2b begins with a name voice command, and then progresses to a verbal command at a first level, and a shoulder shock at a first level. This is then followed by a vibration stimuli, the patient's name being called, a shoulder shake stimuli and a biceps shake stimuli. This stimuli is then followed, if not successful, by a verbal command first level shock, that can then be followed by a secondary level shock, along with other various stimuli. Finally, the fail safe stimuli occurs if none of the precious stimuli levels served to waken the patient.

FIG. 4c illustrates a third different CAST type set of stimuli that may be delivered to a patient, as alternatives to the first and second CAST shown in FIGS. 4a and 4b, respectively.

The sleep disordered breathing (SDB) treatment device 10 of the present invention works as an interactive rehabilitative robotic utilizing operant and respondent conditioning that recruit's the active participation (besides wearing a device) of the patient in the management and control of their Sleep Disordered Breathing.

The device will simulate the human interaction of a lovingly concerned and infinitely patient and non-critical bedmate and personal “ICU sleep nurse”, but will have the machine's advantage of full time alertness. The device includes a multi-sensor input whose microprocessor determines, very early on, when to interrupt or intercept snoring, hypopnea or apnea promptly before a small burst or series of events occurs. The majority of OSAH episodes come in series, and most apneas occur as several exhales progressively lower the functional residual pulmonary reserve and begin at the end of expiration. See Morelli, Arabi, et al., Am. J Resp Crit Care Med 155:A419, 1997. The shallow breathing of Cheyne Stokes Respiration would also be promptly terminated by the device 10, thus preventing the resultant hyperpnia and desensitizing hypocapnia from continuing a vicious destructive cycle for those with the usual CHF. The system also promptly terminates any detected central apnea.

A selected series of cues and stimuli, referenced to herein as a CAST (cues and stimulus train) when initiated, renders a series of stimuli to the patient until snoring either stops or decreases very significantly, or a very adequate inhalation occurs as indicated by sensors. Several different CASTs will be programmed into the main processor (CPU) of the device for the CPU to use, as it will act like a PDA program. At that point the system “resets”, but storage of data, timing and analysis of the response to that CAST is also set. The probable main inputs are simple snore piezo transducer, upper chest expansion (strain gauge, pneumatic tubing, or other), and actual breath sounds mike (probably filtered below 200 Hz to eliminate vascular sounds), HR and an attitude monitor But other possibilities as required or desired are included in envisioned embodiments.

One, two or three variable (and very simple and inexpensive!) muscle pulse stimulators remain poised to deliver tapping, pushing or actual shaking movements to the patient's shoulder and arm. The stimulators will probably deliver electrical stimuli in 2 to 250 Hz range or more, with strength up to 80 to 100 milliamps or more, also variable in pulse frequency, duration, interval spacing, wave form and number of pulses within each burst. Rechargeable DC voltage of 3V, 6V or 9V anticipated. Recharging should be done by using a jack for input from DC charger.

The muscles that are directly stimulated by the electrical pulses used have never been tried for SDB treatment and totally avoid the anterior neck. The electrodes are externally applied. The suprascapularis and trapezius are stimulated by placement of one electrode contact pad at the lower part of the posterior lateral neck on the upper part of the trapezium, and the second electrode on the inferior portion of the contiguous suprascapularis. Pulsing can be timed and strengthened to shake the shoulder from mildly and slowly, to rapidly and forcefully as the system ramps up when the patient doesn't respond to the specific CAST which was used for that event.

The main CPU module is placed over that same scapular within a velcroed shoulder and upper chest adjustable “harness” having a hooked eye (Velcro) fastening belt with the electrodes being direct or hard wired to the CPU. An example of such a harness is shown in FIGS. 1 and 2, and is generally similar in design and configuration to the Sharpe Image Magnetic Therapy harness, model No. HF830, available from The Sharper Image Corporation of San Francisco, Calif. The harness will also serve to securely hold down or contain the electode pad over the suprascapularis. The snore and breath sounds sensors are to be placed on the upper anterior chest wall below and midline to the clavicle (all preferably on the right). with appropriate gain and filtering, unless we must use the lower trachea.

Preliminary investigations indicate that lateral tracheal placement of those small sensors is avoidable, and the upper anterior chest wall provides reliable detection of snoring. This snore sensor is also secured on the chest by the Velcro harness. A small microphone is also connected to the CPU, and can be placed on the anterior chest or on the lateral subaxillary chest wall. The microphone is also directly wired to the CPU and secured by the harness. An attitude monitor for determining the patient's sleeping position (e.g. on back, on side, etc.) is also placed preferably over the scapular as opposed to the arm, as such placement helps to discourage supine sleeping which promotes snoring. Preferably the attitude sensor is made a part of the CPU containing main module. The sensor used to monitor upper chest expansion is either built into the harness, or is separate, but is securely held down onto the patient by the harness.

Two other pair of electrodes are placed on the biceps and triceps respectively. This “arm” unit module is held onto the upper arm with a similar strap using a hook and eye fastener, and receives its directions wirelessly (regular RF, Bluetooth, or other) from the CPU in the main module placed over the shoulder. The shoulder harness may also be constructed to include an adjustable sleeve to hold down the arm stimulator pads.

Alternately the CPU containing main module could be on the arm and communicate with the shoulder stimulator wirelessly. However, it is believed that it would be more cost effective to hard wire the snore, breath and upper chest expansion sensors and shoulder electrodes directly to a CPU held over the scapular with wired connections on under or over surface of the “harness”. When the biceps is stimulated in coordination with the shoulder stimulator we obtain a shaking of the patient that simulates human intervention of the type associasted with an aroused bed partner in the patient's snoring or hypopnea-apnea. If the electrodes are properly placed the shaking or pushing sensations are totally painless, although the CPU could quickly add a pain-inducing element if that were helpful to the conditioning process.

The differing CASTs start at about 3 to 10 seconds into an “event” depending on the CAST sequence used. and ramp up quickly to moderate or strong and terminates at very strong in the shoulder and biceps, and to outright shocking strength (mild to very strong) over the triceps. This is purposeful and is part of the aversive operant conditioning process to fully increase the patency of the upper airway, as one gains with a fuller understanding of how the system works.

The validity of employing a “humonoid” stimuli portion is rooted in studies that indicate that remaining with a bed partner extends life in OSAHS patients. See R D Cartwright in SLEEP 1987; 10 (3)

The CASTs begin with vibrations, like from a cell phone and choices of “ring” again like a cell phone. The voice chip then paces in verbal commands wirelessly to an earphone so that the unit is silent to any bed partner, yet can get very loud to the patient if it needed to, to enhance the conditioning process and terminate events. The verbal communication include such phrases as “stop snoring”, “breath deeper”, “take a deep breath”, “turn over”. etc. The chip could be programmed to recite commands in many different languages, and easily interchanged for various countries and preferences.

The patient's name is actively used by the device to enhance the conditioning process to protect breathing and oxygenation. The device uses a re-programable small digital recorder that allows the patient, or their bed mate (preferably), or a friend or their physician (not preferable except, in some cases) to press a recessed button and announce their own name into the unit, much like a digital phone answering machine. The embedded program will then variably inject their name, at the beginning or the end of probably every third or fourth use of each verbal command. Repeating their name every time decrease or desensitizes the patient to this tool. This turns out to be a very powerful yet mild super-stimulus and is a very important element in the whole reconditioning process when employed appropriately.

The digital recorder also allows the name to be changed to another nick name, or to be re-programmed with a new name for a new patient. A “re-boot” button also will erase all auto-adjusting the old unit did to customize itself and restart from scratch the basic program for the new user. Existing digital technology can be employed to accomplish this. In tests conducted by the Applicant, after only a few hours of conditioning patients were already “subconsciously” anticipating the up-ramping of the cues and stimuli and taking a proper, unobstructed breath just before the stimuli were increased to the higher power to induce uncomfortable muscle contractions and or stinging or painful shocks.

For example, one patient who had Cheyne Stokes Respirations, he promptly prompted halted his shallow breathing after two hours of conditioning in response to just a mild ring, or “Russ, breath deeper”. Over time, the patient aborted the shallow breathing thus avoiding the hyperpnea after only the mention of his name as a stimuli. The CSR did not disappear in the 2 nights during which the experiment was conducted within the prototype wherein the Applicant provided the “artificial intelligence”, but just continued minimal intervention with an “auto-adjusted” mini CAST kept the patient's disordered breathing in control.

A purposeful “fault” mode is programmed in to the device to further enhance conditioning to keep the entire upper airway more patent even in deeper sleep, somewhat like a semiconscious “sentry vigilance”. This “fault mode” occurs approximately every 20^{th} or 25^{th} initiation of a CAST due to sensor input of an event. In the “fault mode” there is a very quick ramp up to strong and a shock. This quick ramp up speeds the response to earlier, milder, less arousals causing elements of the next 19 to 24 CASTs.

A second type of “fault” mode is also employed. In this second fault mode, the equipment allows an event to continue for 20 or 30 seconds and then intercedes. If any mild hypoxia develops in this time then the adequate breath taken when the event ends is a strong positive stimulus to enhance opening of the airway. In uncontrolled apnea-hypopnea this “reward” becomes blunted by baroreceptor and muscle fatigue.

A daytime practice mode is used as part of the conditioning system as an essential part of the conditioning process, and part of the patient's involvement in making the process work for them better. Before patients are allowed to go to sleep the full train of warning cues and muscle stimuli should be demonstrated to the patent. I was going to use if they weren't breathing properly. This demonstration includes having the patient brace themselves for the strongest muscle contractions and especially the actual shocking to their arm and louder verbal commands including their names. Unfortunately, a variable strength shock was not available for this testing sequence, although one will be incorporated into the finished product. The shock experience was helpful because it forewarned the patient of what they are trying to avoid while asleep. As a failsafe reserve, the patient will be shaken hard and shocked if they do not abort an event before the CAST times out.

Surprisingly, a relatively strong shock is not as detrimental to a patient's sleep as one would expect, as none of the patients were consciously aware that I set off the shocker on them, nor remembered it in the morning, although the shock always served to cause the patient to either stop snoring or begin breathing deeply. A relatively stronger shock is needed for the failsafe level that absolutely will awaken the patient if the sensors and timer indicate that this is necessary.

Interestingly, the strongest of the two little AAA battery-device pulse stimulators, was in it was strong enough to contract the trapezius and suprascapalaris to cause sustained extension of the neck, which is desirable if the patient is in a lateral sleeping position, with his/her neck over-flexed thereby, causing compression of their already over compliant airway. The awake time practice mode is set to give an abbreviated combination of CAST sequences while the patient simulates snoring or stops breathing and thereby allow the cues and stimuli of the CAST to be executed.

When awake and in the practice mode, the patient “resets” the device by breathing deeply through their nose using a special “internal yawning” type of oro-pharynx dilating, tongue and jaw protruding maneuver that is taught to the patient. The patient repeats this “awake practice” for 5 to 10 minutes daily, even allowing the CAST to terminate at actual stinging or shocking to their arm before breathing deeply. The practice mode shock is only set to its lowest level. However, when the patient is asleep, especially in the second half of the night, the working unit must deliver a stronger shock. The processor is programmed to automatically escalate the intensity of the shock as the sensors detect the frequency and timing of poor control at lower levels of shock strength.

All levels of auditory, muscle contraction and aversive stinging and shocks will have strength adjustments available for the patient and or therapist to set. The “internal yawn” entails simulating a yawn while pushing the tongue against the upper front teeth and hard palate firmly, while flaring the retro palatal and retroglossal throat open, and breathing deeply through the nose at the same time. This exercise strengthens the tensor palatini, pharyngeal dilators (especially for lateral dilatation of glossopharyngeal space), all the muscles that elevate the hyoid arch and the genioglossus.

This interval yawning exercise is an important basic element in the process of the present invention since it is not yet believed possible to artificially stimulate the body into recreating the complex innervation that the hypoglossal nerve and recurrent larygeal nerves provide to flare open the upper airway as this “internal yawn” does. The tongue must both contract and extend at the same time in different parts to form itself to open the airway. It comprises a set of very unique movements that are only controlled by the brain can accomplish efficiently. One of the goals of the present invention is to hyper-condition that global control, and to accomplish the conditioning, that with progressively fewer EEG arousals as time goes by. The exercise replicates the manner in which OSAH episodes terminate naturally, although natural termination usually only occurs after harming the patient through hypoxias, severe arousals (up to awaking gasping and choking), muscle fatigue and desensitization of the baroreceptors and probably the Respiratory Pattern Generating Nucleus (RPGN). The exercise strengthens tone in the area to a higher level and strengthens all those “flabby” muscles that are part of their more collapsible airway.

The practice mode further conditions the “internal yawn” response to occur in reaction to the milder and earlier elements of the CAST system stimuli of the device while the patient is sleeping. In addition to actually putting on the device for practice, the patient must do the same dilator exercise a few times a day several times in a row to fully increase the strength of these muscles by isometric conditioning so that as waking vigilance is lost during sleep the resting tone is higher than when the patient started using the device. Adding to this is the actual aversive training the device provides during the practice mode and the conditioning that reinforces the practice exercises and responses that occur while the patient is asleep.

For best results with the patient, the “internal yawn” should be demonstrated to the patient to show that the patient that it raises the hyoid arch and advances the mandible which is the object of two of our available surgical modalities. It also fully dilates the oropharynx, tenses the tensor palatini very dramatically and reforms the tongue to maximally open the upper airway. Interestingly two studies were published that show that conditioning goes both ways from sleep to awake and vice versa. See Ikeda and Morotomi in SLEEP 19(1):72-74 and SLEEP 20(11):942-947 1996 & 1997 respectively, and this is partially how to reinforce this whole process.

A patient's use of the device 10 is expected to change the patient's Pcrit so that it would also take a higher negative pressure to collapse the upper airway. Even after collapse of the airway (failure) the device will not let hypoxia develop to any significant extent as the stimuli provided by the machine will help to cause the patient to begin normal, non-disordered breathing.

As part of the therapeutic regime, the patient is strongly encouraged to lose weight, exercise, and attend to upper airway allergies in order for the patient to get the system to work most efficiently for them.

The different CASTs might be called “melodies” as they very in the sequencing and timing of cues and stimuli. The processor evaluates the quickness in terminating an event for the different CASTs. Insufficiently effective combinations of stimuli are decreased in frequency, and very effective CASTs are “played” more frequently. An example of a CAST could be as follows: at 3 to 5 seconds into an event, a vibration stimuli starts mildly; at 8 sec., an auditory stimuli comprising a ringing sound play into earpiece; at 10 sec. The electrical stimuli are triggered to shake the shoulder and/or arm starts followed promptly by verbal command with or without patient's name depending on previous “playing” of that CAST. At 13 secnds, all of the above-referenced stimuli are given again to the patient but at a higher intensity level. At 15 sec. final warning set of stimuli are given to the patient ramp up prior to actual low level stinging or shock sensation. Ramping escalates if proper breath is not taken by the patient.

It is anticipated that 9 to 10 different CAST melodies (variations) will be employed. Further testing will be performed to determine which CASTs result in less arousals and best response time, and which induce the best conditioning reactions. The painful stinging or shocking stimuli, as stated before are important as conditioning tools, which is why they are paced in occasionally during the purposeful “fault” mode, and why they are included in the “practice” mode. The hyper-conditioning of the full reflexive opening of the upper airway by global CNS control, and the increase resting tonicity of all the musculature involved is the method utilized to achieve the primary goal of preserving a healthy O₂ saturation with the least number of clinically significant arousals.

To maximize the effectiveness of the treatment regime to the present invention, it is important to recruiting the patient's cooperation and compliance, by encouraging the patient to make lifestyle and dietary modifications that will, themselves, help to reduce sleep disordered breathing. These modifications include such things as ie., weight loss, exercise, allergy control, daytime practice within the device, dietary modifications and sleep hygiene, etc. These modifications help to make the device and method of the present invention work more efficiently for the patient.

To date, the most frequent CAST or “sequence” that has been used on the test subjects was: (1) a warning beep of 3 seconds; followed by (2) added low verbal command (with or without their name—to avoid desensitizing response to name); followed by (3) addition of shoulder shaking only; and (4) repeated command louder. In most, the patient would already have either stopped snoring or started a deep breath by the time that the repeated command was given.

If not, then intensity of shoulder shake was increased and arm shake was added. If breath didn't normalize, moderate to stronger shocking was then started. As mentioned before, none of the patients was consciously aware upon awakening in the morning that the shocker had been employed to deliver a shock to them. In one case, the full shocker was employed on a test patient about 15 times without the patient remembering anything in the morning. Interestingly, this patient would likely benefit from this present invention, as the patient currently uses a CPAP machine, which he both hates and usually removes from his face in the early morning, which is the time when SDB events are more likely to occur. Additionally, unattended to medical problems, that contribute to his SDB, it is believed that the more holistic approach employed by the present invention would likely be especially beneficial to this subject.

Presented below is a summary of some of the key, unique features of the device and method of the present invention.

A. The device of the present invention comprises an externally applied non-CPAP, maskless, strapless and collarless electronic and mechanical system that acts as an interactive rehabilitative robotic device that facilitates a holistic approach to promoting upper airway patency and responsiveness; to quickly intercept or terminate all significant Sleep Disordered Breathing (SDB) events; and to prevent destructive hypoxias and their resultant hypo & hyperventilations, hyper & hypocapneas, bradycardias & tachycardias, blood pressure alterations, muscle fatigue, edematous palatal inflammations, and arousals with disturbed sleep architecture, in patients with Obstructive Sleep Apnea Hypopnea Syndromes (OSAHS), Upper Airway Resistance Syndrome (UARS), Cheyne Stokes Respiration (CSR), Central Apnea and even “simple” snoring.

B. A device accomplishes these goals by enhancing Global (CNS) Control and the coordination of all the factors that maintain effective respiration, including preventing baroreceptor fatigue and enhancing the responsiveness and tonicity of all the muscles that maintain upper airway patency during all stages of sleep in SDB patients.

C. A device includes a microprocessor that analyzes its multi-sensor inputs to detect faulty or obstructed breathing and is capable of forwarding commands to various output devices, including trains of systemized, precisely timed and coordinated cues and stimuli (CASTs), which cause the patient to respond by breathing properly, thus terminating the disordered breathing event registered by the processor. Because the CASTs differ from each other in content, order and timing they could also be considered “melodies.”

D. The device and system accomplishes its reductino of sleep disordered breathing through operant (Skinnerian) and respondant (Pavlovian) conditioning carried on during both the sleeping and waking state, by including a daytime or evening practice mode for the patient. Positive reinforcers (e.g. positive verbal statements and CNS relief by reinitiating of adequate breaths) and aversive (negative) “reinforcers” (e.g. very strong muscle contractions, loud commands and warning signals, stinging sensations and mild to strong shocking) are employed together in this process.

E. The device has a robotic function that does not attempt to do the complex work of breathing for the patient or directly stimulating the muscles that maintain patency of the upper airway. Instead, the system simulates the actions of a human sleep mate, vigilantly monitoring the patient through sensors and intervening by tapping, nudging or shaking their shoulder and/or arm and giving them direct verbal commands to correct their breathing disorders.

F. The device uniquely allows a patient's name to be inserted into its programming by containing a small re-programable digital recorder, so that the verbal commands are capable of employing the patient's name which helps to make those commands more potent and effective. Thus, the system acts even more like a personally assigned “ICU Sleep Nurse”. Combining the personalized voice commands with shoulder and/or arm shaking brings in the humanoid robotic aspects of the design, which then can combine with standard electronic interventions.

G. The digital recorder is easily re-programmable to enable the name of the patient to be changed, and the new name inserted into the verbal commands program. This changeability allows a different nickname or voice to be added, and permits a single device to be used with a series of many patients.

H. The device provides a humanoid intervention that can also extend the head and neck of the patient to correct an overflexed, airway obstructing position, by initiating strong sustained contractions of the trapezius and suprascapularis muscles instead of using tapping or shaking electrical pulsations to those muscles.

I. The device uses the proprietary algorithms (CASTs) to systematically blend the human-like interventions with non-human like interventions of cues and stimuli, such as vibrations, rings or buzzers, and aversive stimuli, such as stinging and or shocking sensations.

J. The device contains a processor that analyses the speed and quality of the patient's responses to the various CASTs used for different types of events, and through artificial intelligence, selectively favors those that work best for the patient, and reduces playing of those that are less effective. Therefore the embedded artificial intelligence molds the system to work better for each individual patient, thus adding another humanoid intercession to the system's abilities.

K. The present invention provides a device that can easily be set to re-boot and start all over as a “new device”, either to start from scratch for the same patient, due to changing clinical conditions or problems, or be re-programmed for a series of new patients who use the particular device after it is no longer being used by the first patient.

L. The present invention provides a system that is especially applicable for use as an alternative therapeutic agent for those in patients who cannot comply, stop complying, or refuse to comply with any form of Positive Airway Pressure device and their various nasal, or nasal-oral interfaces. This group of patients includes those persons who refuse either surgical interventions or to be fitted with oral devices. The present invention will provide such patients with an alternative device capable of providing positive therapeutic benefits.

M. The present invention provides a device that accounts for a patient's sleeping positions, as it can keep track of positions that lead to or increase airway obstruction and can help to hyper-condition the patient to avoid those positions, unless such positions make drastic improvements in lessening the severity of patient's SDB, or unless such positions acclimate healthfully to those positions. By contrast, PAP devices encourage or enhance sleeping in the supine position to avoid displacing the interface, since the pneumatic “splint” (PAP) artificially holds the airway open for them. Sleeping in the supine position is usually the worst sleeping position for these patients. If a patient becomes used to sleeping in the supine position, and then removes his/her PAP equipment in the early AM, as a large percentage of patients do, or stop compliance totally, the patient may have worse or more frequent O2 de-saturations than before starting treatment.

N. The preferred embodiment can be designed to be wearable during the day or kept in a manner that is basically invisible to other people when worn under a shirt or pajama, unless an embodiment utilizing a small oral-nasal canula is employed. This “invisibility” helps overcome the reluctance, embarrassment and vanity that accounts for some patient's lack of compliance with PAP devices and interfaces. This reluctance to be seen in a PAP device is especially true for younger adults, single patients that are dating, and any patient that is reluctant to look like an “air-force pilot” or a “sick patient” while in bed for whatever reason.

O. The device of the present invention does not require facial or head straps, or uncomfortable nasal or oral-nasal interfaces, that also can leave unsightly pressure marks on the face or messed up hair in the morning. These aesthetic insults are a factor in non-compliance in some women patients.

P. The device that does not need to be removed if the patient gets up, walks around, or changes the place they are sleeping in. Patients can sit in their living room and watch television or read, etc. and not worry about falling asleep without having their SDB treatment device on and activated. They can perform the same activities in their beds and not have to be wearing an interface that interferes with or interrupts these activities as they fall asleep. These are further factors that could increase on going long-term compliance.

Q. The device contains a voice chip for verbal commands that can be easily exchanged allowing the device to use different languages appropriate for the patient or the country in which they are deployed.

R. The device of the present invention does not use implantable electrodes, and does not directly stimulate the hypoglossal, recurrent laryngeal or vagus nerves, thereby reducing many regulatory difficulties when compared to more invasive devices.

S. No embodiment of this system requires a cumbersome, unsightly or embarrassing or “demeaning” collar to be worn around the patient's neck.

T. The present invention promotes an interactive and rehabilitative process as described above by having a practice mode that can be used during waking hours several minutes a day, especially when first starting to use the equipment. The use of the device in the practice mode greatly enhances the patient's responsiveness to the CASTs used by the device during all sleep stages and incorporates and encourages the patient to be actively responsible and involved in their SDB therapy.

U. The active involvement of the patient is achieving his own wellness is further promoted by the system's emphasis teaching the patient how attendance to their health conditions and life style habits can make the device work more efficiently for them, and positively affect their future health. Issues of clearing the nasal passages, treatment for allergies, weight loss, cardiovascular conditioning through some exercise, sleep hygiene, stress management, medication management, alcohol or cigarette use all become much more relevant to the patient which thereby helps the system work better for them.

V. Except for patients using nasal CPAP, who must keep their nasal passages clear, almost all other aspects of health promotion and disease prevention strategies may tend to be ignored in PAP compliant patients, with them thinking they are “doing enough” by just using their PAP equipment. Engaging in less healthy dietary habits, gaining weight, getting further out of shape, using too much alcohol or sedative, not keeping their nose clear, getting too little sleep, etc. will cause the electronic system of the present invention to ramp up to deliver more frequent and more disturbing CASTs, thus signaling the patient that their transgressions are exacting “a price”. Thus this system can serve as a tool for ongoing recruitment of the patient's efforts to manage their broader health issues, not just their SDB related issues, thereby making the device a potentially valuable tool for promoting overall patient health.

W. The device of the present invention includes an LCD display on the main module that gives important messages about the equipment's condition such as power reserve, sensor functioning, etc., and also displays alerts of poorer sleep and breathing, and/or positive statements when the data analysis shows improvements (fewer events recorded). These comments communicated through the display are part of the overall “conditioning and recruitment” process of the system. If the equipment is performing satisfactorily and the patient shows no worsening or improvement on the previous night the LCD can be prompted to simply say “no messages”, which could be construed as a reward or a punishment with different patient personalities.

One feature of the present invention is that this system can contain a booklet of health recommendations that is provided to the patient and the reasoning behind the recommendation, that is preferably prepared by a panel of qualified experts in sleep medicine, and intended to further educate and motivate the patient to utilize the system more efficiently. Much of this advice contained in the booklet is intended to supplement the information provided by the patient's physician and will thus enhance and re-enforce the physician's recommendations. FAQs about how the device actually works can also be included. This instruction booklet should frequently remind the patient that their health is their responsibility, not their therapist's, and should state that no one is expected to be “perfect”, but that any further improvements they could make in their life style is to their great advantage in the long run, with or without using the equipment.

X. The daytime practice mode of the device consists of activating the “practice mode” switch and putting on the shoulder harness and armband modules. The patient then either simulates snoring or holds their breath and lets the unit play through some abbreviated practice CASTs, terminating their simulated events after a few to several or more seconds, by either ceasing their snoring or taking a deep breath. Sometimes the patients will be instructed to terminate the CAST after low verbal commands or mild shoulder and/or arm shaking, sometimes the patients will be instructed to let the CASTs ramp up stronger before terminating the simulated event, and even to allow some CASTs to ramp up to mild arm stinging or shocking sensations (level one or even two, if they can tolerate it) before terminating the event. This awake time conditioning carries over to their sleep time and improves responsiveness to the various cues and stimuli. All events are terminated by breathing deeply through the nose while doing a “Concealed Yawn”.

Y. The “Concealed Yawn” exercise of the present invention is one way the patient improves the sleeping and waking tonicity and coordinated reflexive responsiveness of the approximately 20 muscles involved in controlling upper airway patency. The tensor palatini, pharyngeal muscles, genioglossus, the geniohyoid and other muscles elevating the hyoid arch all become strengthened and more quickly responsive to the device's cues and warnings. Practicing this “isometric” becomes another holistic recruitment of the patient's cooperation with improving control of their SDB. Interestingly, this maneuver entails relaxing of the levator palatini, and muscles that lower the hyoid arch (sternohyoid and thyrohyoid), a process believed to be too complex for science to recreate with current technology.

In order to perform the “Concealed Yawn”, the patient pushes the tip of the tongue firmly up against the upper teeth and hard palate with the teeth slightly parted, while the patient flares their nostrils and oropharynx open with a “fake” yawn in a manner similar to trying to conceal a real yawn by keeping one's lips closed while yawning. The patient should thrust their jaw forward, pull down the soft palate and tense the pharyngeal constrictors and depress their posterior tongue while breathing deeply through the nose. This exercise is done several times in a row as a “isometric” exercise, and the patient is asked to repeat this series a few times a day, at first, and to always terminate their simulated SDB events during the practice mode (#24) by breathing in deeply through their nose using this “Concealed Yawn” maneuver. Preferably, the technique is to be demonstrated to the patient by the therapist to enable the exercise to be fully appreciated by the reviewer and the patient.

Z. The Applicant's device does not require practice of the Concealed Yawn or the practice mode to prevent significant SDB events and maintain near normal O₂ saturations. However, practice does help reduce response time and reduce arousals, and emphasizes the importance of the patient taking some further important steps in managing their disease.

AA. The modules, harness and armband of the device can be made small enough to fit younger children, pre-adolescents and teenagers. This would be especially valuable for children who absolutely refuse to comply with PAP devices and interfaces, causing arguments, fear and dysphoria in the family.

AB. For very young children that only use an apnea monitor and alarm system, the device not only allows the parents to sleep more restfully and assured, but also treats the child's apnea by the same conditioning responses as in the adult. Very young children would not be expected to use the practice mode, or practice the Concealed Yawn isometric.

AC. The ease with which the device can be used helps to promote the compliance of the user with his therapeutic regime. Even with adults stress is greatly reduced in the family and with a spouse if the patient complies with his treatment regime by using the device. Once the diagnosis of SDB is made and the seriousness of the disease and its complications is explained to a spouse, worry, fear and discord in a family if the patient refuses to comply as is more common than not with PAP device therapy. The patient is much more likely to sleep with their mate in the same room if using this novel mechatronic device, as opposed to PAP therapy where a spouse may find the noise of a PAP machine disturbing (or use the noise of the PAP machine as an excuse to sleep elsewhere).

AD. The preferred embodiment of the device can include a voice chip that also has positive motivating statements embedded, that occasionally play at a low volume during times of quiescence between intercepted events, while the patient sleeps and the position monitor indicates that they are not up walking around, going to the bathroom, etc. Examples of such motivating “semi-subliminal” statements include (but are not limited to) (a) “you will work hard to be at a healthy weight”, (b) “you will try to exercise and be in better shape”. These statements could be embedded in the main processing unit's voice chip, and an enable or disable switch for this modality could be added to the control panel of the main module, or this could be an optional added chip only for patients who want this in their unit.

AE. The main sensor inputs analyzed by the microprocessor to accomplish the device's purposes include, preferably, a piezo-electric snore monitor, a small breath sounds microphone, a heart rate sensor, an attitude monitor, and an upper chest expansion sensor (either strain gauge, piezo, pneumatic tubing or other), all of which are well known in the art and commercially available. Additional options that can be included in various embodiments of the design can include an EMG muscle tension probe (either on the inferior portion of the sternocleidomastoid or the lateral intercostal area above the diaphragm), a thin non-obtrusive oral nasal airflow canula (such as the canula sold by ProTech) with the transducer in the shoulder module, an oximeter (with probe on finger, earlobe, or toe), accelerometer wirelessly transmitting from the lower leg, blood pressure monitor, one or two channel EEG leads, and including one EOG lead that simultaneously serves as one EEG lead, and can even include a LED for photic stimulation if desired (as a cue in a special CAST). This last combination sensor would have to be proprietarily designed since it is not commercially available. Any of these options can be included in the main embodiment if found necessary or advantageous during design or clinical research trials.

AF. The present invention can employ MEMS (micro electrical mechanical system) technology to provide some of the system's sensor inputs, and even be incorporated into the shoulder harness material itself to reduce the bulkiness or inconvenience of too many sensor connections.

AG. The device can be constructed using off-the-shelf sensor inputs with the main module containing prefilters, and an analog to digital converter. Alternately, digital acquisition may be used followed by digital filtration prior to analysis and digital storage (DDD technology) for greater accuracy, if required or desired. Standard 50 Hz and 60 Hz filtration will remove powerline frequencies. Appropriate filtration to insure accurate detection of HR, snore vibrations, breath sounds, etc., is included in the system.

AH. Three electric pulse stimulators will be used in the device, including one in the main module over the scapular area, and two within the armband module. The arm stimulator that contracts the biceps may be identical to the one whose electrode contacts will stimulate the trapezius and suprascapularis muscles. The CPU utilizes a multiplexer so that if a particular CAST is stimulating the shoulder and arm at the same time, the contractions will be simultaneous and coordinated (instead of in opposite directions) to produce the type of shaking that a human intercessor using two hands (one on the shoulder and one on the arm) would produce. These two pulse stimulators can vary from 2-250 Hz or more, with strengths up to 80-100 milliamps or more, with variable wave form choices, duration, interval spacing and number of pulses within each burst as desired based on further design trial testing. The device may include routine electrical muscle stimulators (EMS), modified transcutaneous nerve stimulators (TENS), or interferential (IF) stimulators. IF stimulation using high frequencies (2000-4000 Hz), these high frequencies can be biphasically matched to carry packets of lower frequency (50-200 Hz) to allow a deeper muscle stimulation with less potentially annoying skin sensory nerve sensations. However, other choices can be employed other than those set forth above.

AI. The stimulator with electrode that is intended to contact the triceps (or an embodiment with one electrode on the mid-biceps and one on the mid-triceps) can be an aversive stimulator that is simpler, and acts more like a variable intensity dog training shocker. The stimulator may also include a vibration mode to import extra warnings to the patient that the arm is about to receive an aversive stimulation unless breathing returns to normal within a couple of seconds. Its main function is to provide the aversive stimuli, varying from stinging up to mild brief “nicking” pulses that may be between 1/100-1/400 th sec. in duration, to stronger continuous bursts of painful shocks. Earphone warnings would be simultaneously transmitted to the patient, as would increased shoulder and/or arm shaking, prior to this. These aversive stimuli are a necessary and integral part of the system's design to facilitate reconditioning of SDB events and maintain sleeping vigilance of the upper airway muscular control of airway patency, an good CNS global control and respiratory drive.

AJ. Another aspect of the device is that it includes a failsafe mode wherein a stimulator is used to produce the strongest shocks, guaranteed to awaken the patient in case of a seriously long apnea (perhaps 60 or more seconds or longer but not restricted to this time frame). The failsafe mode also provides a loud alarm that would be audible to a bedmate or a family member. The CPU also issues a loud alarm signal to the patient through the patient's earphone and vigorously shakes the patient's shoulder at maximum strength when the failsafe mode activates.

AK. The earphone for the device can be wirelessly controlled from the main module or can be hard wired directly to the main module. If a patient always sleeps alone, they may decide against using an earpiece, and instead, activate a speaker within or attachable to the main module having its volume set loud enough to be easily heard by the patient. This offers one less impediment to sleep and would be especially helpful with children sleeping in their own rooms, thus avoiding any inconvenience associated with the earphone, such as displacement while turning in bed, or laying with that ear on the pillow, thereby causing discomfort to the patient.

AL. Two purposeful “Fault” modes are programmed into the CPU that further enhances the patient's responsiveness to the various cues and stimuli. The first fault mode occurs perhaps every 20 or 25^{th} detected event (but not limited to these numbers), and purposely ignores the sleep disordered breathing event for a certain preset time, such as 20 or 30 sec., and then intercedes with an ordinary CAST. If any mild hypoxia develops and ventilatory drive increases then the next breaths taken by the patient serve as a metabolic and CNS “reward” for ending the event. In uncontrolled recurrent apnea-hypopnea this reward is blunted by baroreceptor and central drive fatigue. The second type of fault also occurs intermittently and entails suddenly initiating a very quick ramp up to aversive stimuli in response to the event, and perhaps accompanied by the appropriate verbal command for that event, such as “take a deep breath”, or “stop snoring”. This quick ramp up fault mode improves response time to the next 19 to 24 CASTs, thus further reducing the micro arousals that CASTs may cause, if played out and ramped up.

AM. The optional use of the actimeter, perhaps just intermittently, may be desired to differentiate arousals possibly caused by the device's interventions from those caused by a Restless Leg Syndrome (RLS), or a Periodic Limb Movement Syndrome (PLMS). The actimeter is designed to be in communication with and under the control of the main processor CPU module.

AN. The device of the present invention can be modified to work within or be utilized in conjunction with various PAP devices and/or their interfaces, to enhance the same Global Control reconditioning processes to reduce the severity of disease in SDB patients, perhaps reducing autoPAP, arousal inducing, required pressure ramp-ups. These embodiments would also help in patients that are only partially complying with PAP therapy of planning to, or who are determined to be likely to discontinue use of their PAP device.

AO. The electrode pads used to deliver impulses from the stimulators can be of various types, including self-adhesive silverized, or carbonized rubber, or non-adhesive types utilizing less irritating conductive gels. Specially made pads with Velcro backs may be used so that proper placement over the target muscles for the each patient is maintained each night by adhering to the inside portion of the shoulder harness or the arm pad. The aversive triceps stimulator electrode contacts may be part of the underside of the arm module and contact the skin through two holes in the armband. See FIGS. 1 and 5a-5c.

AP. The device of the present invention can be modified to add additional outputs and additional reporting capabilities to better adapt the device for use by trained technicians during attended sleep studies. This clinical trial version can be designed to transmit the multiple sensors' outputs wirelessly (RF, BlueTooth, WAN, LAN, IT Ethernet Modem or other) along with video cam images of the sleeping patient. The patient's embodiment will also incorporate a wireless receiver allowing the researcher to control the patient's stimulators and earphone, by transmitting different CAST algorithms and observing the patient's responses in a montage display format on the receiving computer's screen. EEG, EOG, EMG, oral nasal airflow transducer and leg actimeter tracings can be utilized with this testing mode embodiment, and incorporated into the device either as options or separately applied commercial PSG equipment (as unobtrusive as possible). All sensory data and responses to various trial CASTs can be recorded and analyzed by the researcher and/or medical practitioner.

AQ. In the preferred embodiment of the system the microprocessor will either store its analysis reports on a compact flash (SMART) card, or directly transmit these reports by IT to the therapist's computer and/or the company's main frame system for further analysis, research or documentation. If necessary, these reports can be transmitted to third party payers who want documentation of effectiveness or compliance with equipment use.

AR. One feature of the present invention is that it results in the early termination of SDB events. Early termination of SDB events prevents the clusters of apnea-hypopneas from occurring which, by their very O₂ saturation depleting nature, drive the untreated patient's O₂ level down more quickly with each successive obstruction. Arousals caused by recovering from these more intense desaturations are much worse, even causing awakening, compared to the micro arousals the device would cause during early termination of an impending cluster.

AS. The device can also include the use of an intercostals or other muscle EMG sensor, and an EOG sensor may be used to indicate REM sleep. The processor may delay initiation of particular CASTs 10 to 25 seconds, or only use minimal cues during REM with the hope that the patient will respond without terminating the REM sleep phase.

AT. Pure Cheyne Stokes Respirations (CSR) usually diminish or stop during REM sleep (see Lee-Chiong, Sateia, and Carskadon; Sleep Medicine 2002, Henley & Belfus; p. 635). Thus, REM would be best preserved in these patients who suffer from Cheyne-Stokes Respirations. Also the arousals of CSR sufferers tend to occur at the peak of the crescendo-decrescendo pattern, and the device is designed to terminate these CSR events early on by ending the shallow hypercapnia causing breathing, thus also limiting more significant arousals and the hypocapnia inducing hyperventilations. If central apnea occurs in CSR it would also be quickly terminated. Through this vehicle, the device can control CSR very well.

AU. Another feature of the device is that it includes several vehicles for achieving an early detection of snoring. Most OSAHS and UARS events are preceded by significant snoring, which is very easily detected by the equipment, and can be promptly terminated before they worsen. Sudden hypopnea without the prior warning snoring sounds can be detected by the processor's chest expansion and breath sounds algorithm and terminated before apnea can occur. Adjustment of the snore sensor setting can even allow for a certain decibel level of snoring for a particular patient, if that would be helpful in preserving a healthier sleep architecture for them.

AV. The device of the present invention can also contain a disabler of the treatment mode and the device so that the device can be employed and will record and report untreated SDB events. Thus the device can be adjust to serve as a diagnostic or screening device for SDB. Such an embodiment probably would have the oximeter option added. This treatment-disabled mode is useable to evaluate the patient's response to compliance. Reports of these non-treatment nights can be used to encourage or praise life style improvements with the patient.

AW. The device of the present invention also contemplates the use of a less expensive, stripped down version for use to treat simple snoring and mild UARS. Such a stripped down version utilizes only a snore monitor, reduced CASTs, full voice chip capacity with patient's name, simpler intelligence and stimulators. No analysis or reporting functions would be required. Only one module is envisioned, with one muscle contracting stimulator doubling as the aversive stinging, nicking or shocking element also, but not limited to this if so required. This embodiment has the potential to be marketed as a non-medical, OTC device only for its stated purposes.

AX The device comprises an interactive, rehabilitative robotic for the holistic treatment of all Sleep Disordered Breathing (SDB) syndromes, and simple snoring, which system functions like two separate devices to accomplish said treatment, both using operant and respondant deconditioning and reconditioning processes, and motivational processes. The system's treatment mode can be disabled (becoming a third device) turning it into a full SDB home screening device generating only its data report of events. The treatment system uniquely incorporates humanoid intercessions, intelligently blended with various electronic cues and stimuli, to correct breathing faults, and preserve oxygen saturation, without requiring any uncomfortable equipment on the face or anterior neck.

AY. During sleep, the system stops or reduces, and deconditions against all factors leading to, or causing obstructions to, or cessation of breathing, while simultaneously reconditioning Global CNS and peripheral coordination and control of breathing. During waking hours the system's mode can be switched to become a training device, to be utilized by the SDB patient to strengthen all the neurological and muscular coordinations required for normal breathing, by practicing and reinforcing a specific “reflexic” exercise that increases or helps maintain upper airway patency. During such practice, the patients also better condition themselves to more quickly respond, in the same reflex fashion, to the various intercessions the device uses during sleep treatment when an event is detected, thereby reducing arousals from stronger intercessions.

AZ. The system employs the patient's name intermittently as part of its verbal commands, and uses verbal instructions that are specific to the type of breathing fault and position of the patient, with language chips being made available in a plurality of languages so that the patient receives the instructions in his native language. The system can contain a voice chip that optionally contain motivational statements, to be “whispered” to the patient, almost subliminally, while they are asleep, if they elect to enable that function.

BA. The device contains sensors and a microprocessor, that are capable of distinguishing between snoring, more significant airflow reduction (hypopnea), total obstruction, the hypoventilation phase of Cheyne Stokes Respiration and central apnea, and then are capable of adjusting the verbal commands given to the user to give comments that are appropriate for the type of SDB event.

BB. The system is capable of shaking the patient's shoulder gently or intensely, to promote proper breathing and terminate all SDB events which shaking simulates a.humanoid intervention. The system that can extend the patient's neck, thus correcting an over flexed, airway compressing position, and keep the shoulder in sustained contraction, if so desired, which simulates another humanoid intervention. Further, the system that can flex the patient's arm intermittently, and coordinate with shoulder shaking to produce a stronger shaking effect, or could also keep the biceps in sustained contraction which simulates another type of humanoid intervention.

BC. The system is designed to utilize interferential currents, if desired, from its pulse stimulators, to reduce unpleasant cutaneous sensations, while still being capable of utilizing aversive “nicking”, stinging or shocking sensations from traditional one frequency stimulators. Notch filters can be employed to block any generated treatment frequencies, to prevent contamination and distortion of required frequencies for its sensors. Standard sensors and stimulators, or MEMS (micro-electrical mechanical systems) technology can be employed for miniaturization and convenience.

BD. The components of the system are designed to contain modules small enough to be used on very young children, yet powerful enough to also be used on very large or obese adults. Although the preferred embodiment is totally externally applied, variations are envisioned in which very small “bionic neuron” devices may be non-invasively implanted, subdennally or intramuscularly, to replace some of the external sensing or stimuli delivering functions depicted in the drawings and discussions presented in this patent.

BE. The system's humanoid interventions are variously blended into other cues and stimuli to form trains, with several or more CASTs being utilized to correct pathological breathing events in differing fashions. The microprocessor digitally analyzes and stores the speed, quality and duration of responses to various CASTS, and intelligently reconfigures itself through artificial intelligence to increase or decrease the playing of different CASTs for specific breathing faults, in order to function more effectively for each patient. The usual pattern of treatment CASTs is designed to occasionally produce one of two different “fault” patterns. One fault pattern produces a quick moderate aversive shock at the beginning of a breathing event, with no warning cues. The other “fault” purposely allows an event to continue for quite a while, before taking actions.

BF. The system is capable of estimating REM sleep phases from HR, intercostal EMG, and respiratory rate (and the optional sensors) and delays all intercessions for 20 or more seconds trying to allow more REM sleep and patient self termination of events.

BG. The system is designed to have its digital storage on a SMART card and/or uploaded wirelessly through a port, or any appropriate mode desired. Field reprogramable chip technology is employed along with a microprocessor whose entire program can be conveniently altered as desired, and also have its voice chip, or its CASTs replaced to increase the flexibility and adaptability of the device. The system is also designed to permit total monitoring, control and analyses to be performed in a different location from where the patient is sleeping by being capable of transmitting data it obtains at any distance, either wirelessly or by a modem.

BH. The device is designed to be used as a component of a holistic treatment plan. In this regard, the device works even better, with less intercessions during sleep if the patient cooperates with improving their health (weight, etc.), and practices a little during waking hours. The patient is being actively recruited by the system's processes to be proactively involved in treating their SDB condition. Backsliding and other variables will cause more aggressive CASTs to occur on nights with increased events, and commentary on the LED screen the next day will inform the patient of their worse previous night, and ask them to look into the reasons why the backsliding has occurred.

BI. The device is designed to utilize a flip top LED touch screen for easier viewing of simple commentary from the device on weight, compliance, improvements or backsliding, suggestions, etc. Verbal reports from the equipment would be a hindrance and annoying. Very importantly (for most SDB patients), and unique, the patient's weight is entered into the treatment device, and updated weekly or monthly, using the touchscreen, for data entry, and responses to a few simple questions, whose format is designed to maintain and maximize motivation and involvement with treating their SDB more effectively (if possible).

BJ. In addition, a simplified embodiment can be produced that utilizes only a piezo snore sensor and a single module, utilizing CASTs only for snoring, but still employs the same intercessions, and even a practice mode as described for the SDB treatment device. Further, an embodiment containing elements of the system can be incorporated into, or functioning along with auto-CPAP devices allowing them to effectively terminate any central apnea or incipient Cheyne Stokes Respiration episodes promptly. An expanded embodiment of this approach contains most of the capacities of the standard system and can also be incorporated into CPAP devices for use in patients that usually remove their PAP interface in the middle of the night. This embodiment stays quiescent until the aPAP device detects that the patient has removed their interface, at which point the microprocessor in the PAP device initiates all treatment CASTs, through wireless control of the shoulder and arm modules.

BK. A further feature of the invention is that some of the electrode contact pads utilized to deliver pulsations through the skin are novel dual purpose pads containing a small surface area contact, surrounded by a larger area contact, each with their own wire. The same strength pulsation that will cause muscle contraction if transmitted into the larger surfaces, will cause a significant aversive stimulus, without much muscle contraction, if transmitted into the much smaller surfaces. This not only conserves battery power, but requires only one pad placement to be maintained, instead of two for each electrode contact point.

What is claimed is:

1. A device for treating sleep breathing disorders of a patient comprising
a processor for receiving sensor inputs, processing the received sensor inputs and generating comments to output devices,
a first sensor positionable for receiving breathing sounds information emitted from a mouth and nose of a patient,
a second sensor positionable on a patient for receiving breathing sounds information from a patient's chest cavity and
a third sensor positionable for receiving information to the amount of chest expansion of a patient;
a first output device capable of providing an auditory signal to a patient,
a second output device capable of providing an electrical signal to a muscle group of a patient that simulates a human touching event
wherein the first, second and third sensors, and the first and second output devices are operatively coupled to the processor to permit the processor to receive information inputs from the sensors, process the input information to detect the existence of a sleep breathing disorder event, and to generate a command to at least one of the first and second output devices, the command being capable of directing the at least one output device to provide a series of progressively intrusive stimuli designed to condition the patient to terminate the sleep breathing disordered event.
